SEQUENCE LISTING (2 sequences)

SEQ ID NO: 1 - DNA, 42 bp, Artificial Sequence
  Completely Synthetic Nucleotide Sequence
gcaacgattaatatggcgtcgcagccaaattcgtctgcgaag

SEQ ID NO: 2 - DNA, 60 bp, Artificial Sequence
  Completely Synthetic Nucleotide Sequence
gcaacgctcgagtcagtgatgatggtggtgatgctgattcacttcaggcttattcaatat

(19) United States
(12) Patent Application Publication
(10) Pub. No.: US 2009/0012061 A1
(43) Pub. Date: Jan. 8, 2009

(54) A Method of Treating Cancer

(76) Inventors: Timothy A. Blizzard, Princeton, NJ (US); Carolyn A. Buser-Doepner, Harleysville, PA (US); Douglas E. Frantz, Downingtown, NJ (US); Kelly Hamilton, North Wales, PA (US); Myle Hoang, Colonia, NJ (US); Ling Lee, Lansdale, PA (US); Christopher R. Moyes, Hertfordshire (GB); Jerry A. Murry, New York, NY (US); Arash Soheili, Iselin, NJ (US)

Correspondence Address: MERCK AND CO., INC, P O BOX 2000, RAHWAY, NJ, 07065-0907, US

(21) Appl. No.: 11/662,396

(22) PCT Filed: Sep. 9, 2005
(86) PCT No.: PCT/US05/32037

§ 371 Date: Mar. 9, 2007

Related U.S. Application Data
(60) Provisional application No. 60/609,428, filed on Sep. 13, 2004.

(51) Int. Cl. G01N 33/574; C12N 9/99; A61K 31/403; A61K 31/428; C07C 271/06; A61K 31/27; A61K 31/47; C07D 215/38; C07C 39/12; A61K 31/05; C07C 275/28; A61K 31/17; A61K 31/165; A61K 31/415; A61K 31/44; A61K 31/5415; A61K 31/551; A61P 35/00

(52) U.S. Cl. 514/218; 435/7.1; 435/184; 514/411; 514/367; 560/27; 514/535; 514/313; 546/159; 568/719; 514/732; 564/52; 514/598; 514/617; 514/406; 514/344; 514/226.2; 514/418

(57) ABSTRACT

The present invention relates to a method of treating cellular proliferative diseases, in particular cancer, which comprises administering a modulator of the activity of the mitotic kinesin KSP, wherein the activity of the KSP modulator is dependent on the presence of microtubules. It is believed that the KSP modulators utilized in the instant method bind to the KSP protein in a previously unreported manner, since the compounds do not bind competitively with respect to either microtubules or ATP, the substrates of KSP. The modulators useful in the instant methods are furthermore not active against the non-microtubule stimulated activity of KSP. Cellular proliferative diseases that may be treated using the method disclosed herein are, for example cancer, hyperplasias, restenosis, cardiac hypertrophy, immune disorders and inflammation.

### BACKGROUND OF THE INVENTION

This invention relates to a method of treating cellular proliferative diseases, in particular cancer, which comprises administering a modulator of the mitotic kinesin KSP, wherein the activity of the KSP modulator is dependent on the presence of, but does not bind competitively with respect to, microtubules. Cellular proliferative diseases that may be treated using the method disclosed herein are, for example cancer, hyperplasias, restenosis, cardiac hypertrophy, immune disorders and inflammation.

Among the therapeutic agents used to treat cancer are the taxanes and vinca alkaloids. Taxanes and vinca alkaloids act on microtubules, which are present in a variety of cellular structures. Microtubules are the primary structural element of the mitotic spindle. The mitotic spindle is responsible for distribution of replicate copies of the genome to each of the two daughter cells that result from cell division. It is presumed that disruption of the mitotic spindle by these drugs results in inhibition of cancer cell division, and induction of cancer cell death. However, microtubules form other types of cellular structures, including tracks for intracellular transport in nerve processes. Because these agents do not specifically target mitotic spindles, they have side effects that limit their usefulness.

Improvements in the specificity of agents used to treat cancer is of considerable interest because of the therapeutic benefits which would be realized if the side effects associated with the administration of these agents could be reduced. Traditionally, dramatic improvements in the treatment of cancer are associated with identification of therapeutic agents acting through novel mechanisms. Examples of this include not only the taxanes, but also the camptothecin class of topoisomerase I inhibitors. From both of these perspectives, mitotic kinesins are attractive targets for new anti-cancer agents.

Mitotic kinesins are enzymes essential for assembly and function of the mitotic spindle, but are not generally part of other microtubule structures, such as in nerve processes. Mitotic kinesins play essential roles during all phases of mitosis. These enzymes are “molecular motors” that transform energy released by hydrolysis of ATP into mechanical force which drives the directional movement of cellular cargoes along microtubules. The catalytic domain sufficient for this task is a compact structure of approximately 340 amino acids. During mitosis, kinesins organize microtubules into the bipolar structure that is the mitotic spindle. Kinesins mediate movement of chromosomes along spindle microtubules, as well as structural changes in the mitotic spindle associated with specific phases of mitosis. Experimental perturbation of mitotic kinesin function causes malformation or dysfunction of the mitotic spindle, frequently resulting in cell cycle arrest and cell death.

Among the mitotic kinesins which have been identified is KSP. KSP belongs to an evolutionarily conserved kinesin subfamily of plus end-directed microtubule motors that assemble into bipolar homotetramers consisting of antiparallel homodimers. During mitosis KSP associates with microtubules of the mitotic spindle. Microinjection of antibodies directed against KSP into human cells prevents spindle pole separation during prometaphase, giving rise to monopolar spindles and causing mitotic arrest and induction of programmed cell death. KSP and related kinesins in other, non-human, organisms, bundle antiparallel microtubules and slide them relative to one another, thus forcing the two spindle poles apart. KSP may also mediate in anaphase B spindle elongation and focussing of microtubules at the spindle pole.

Human KSP (also termed HsEg5) has been described [Blangy, et al., Cell, 83:1159-69 (1995); Whitehead, et al., Arthritis Rheum., 39:1635-42 (1996); Galgio et al., J. Cell Biol., 135:339-414 (1996); Blangy, et al., J. Biol. Chem., 272:19418-24 (1997); Blangy, et al., Cell Motil Cytoskeleton, 40:174-82 (1998); Whitehead and Rattner, J. Cell Sci., 111:2551-61 (1998); Kaiser, et al., JBC 274:18925-31 (1999); GenBank accession numbers: X85137, NM004523 and U37426], and a fragment of the KSP gene (TRIP5) has been described [Lee, et al., Mol. Endocrinol., 9:243-54 (1995); GenBank accession number L40372]. Xenopus KSP homologs (Eg5), as well as Drosophila K-LP61 F/KRP 130 have been reported.

A number of research groups have recently described compounds of great structural variety as being inhibitors of KSP (see for example: PCT Publications WO 01/30768, WO 01/98278, WO 03/050,064, WO 03/050,122, WO 03/049,527, WO 03/049,679, WO 03/049,678 and WO 03/39460 and pending PCT Appl. Nos. US03/06403 (filed Mar. 4, 2003), US03/15861 (filed May 19, 2003), US03/15810 (filed May 19, 2003), US03/18482 (filed Jun. 12, 2003) and US03/18694 (filed Jun. 12, 2003)). Inhibitor compounds described in several of those applications have been particularly described as binding to the L5 loop of the KSP motor domain. A series of compounds derived from a marine sponge Haliclona sp. have been described as mimicking the activity of a microtubule (U.S. Pat. No. 6,207,403). Those compounds bind to KSP competitively with respect to microtubules and non-competitively bind to the KSP protein with respect to ATP.

Mitotic kinesins are attractive targets for the discovery and development of novel mitotic chemotherapeutics. Accordingly, it is an object of the present invention to provide compounds, methods and compositions useful in the inhibition of KSP, a mitotic kinesin.

### SUMMARY OF THE INVENTION

The present invention relates to a method of treating cellular proliferative diseases, in particular cancer, which comprises administering a modulator of the activity of a kinesin motor protein, in particular the mitotic kinesin KSP, wherein the activity of the modulator is dependent on the presence of microtubules. It is believed that the modulators utilized in the instant method bind to the kinesin motor protein in a previously unreported manner. The compounds described herein do not bind competitively with respect to either the microtubules or ATP, the substrates of mitotic kinesin proteins. Furthermore, the compounds described herein are active only against the microtubule-stimulated KSP ATPase activity and do not show inhibitory activity against the basal (non-microtubule stimulated) KSP ATPase activity. Cellular proliferative diseases that may be treated using the method disclosed herein are, for example cancer, hyperplasias, restenosis, cardiac hypertrophy, immune disorders and inflammation.

### DETAILED DESCRIPTION OF THE INVENTION

It is a discovery of this invention that certain small organic molecules can specifically modulate mitotic kinesin activity only when the kinesin is bound to a microtubule. These molecules however do not act as competitive inhibitors for microtubule binding and for ATP binding. This is a previously unknown mechanism of kinesin (or other motor, e.g., myosin or dynein) modulation. Thus, in one embodiment, this invention provides methods of identifying kinesin inhibitors that do not block the microtubule binding site and the ATP binding site, but depend on the presence of microtubules for mitotic kinesin inhibitory activity.

Such specific modulators are characterized by the fact that they can bind to the kinesin motor protein only if the protein is also bound to a microtubule. In one embodiment, this invention therefore provides methods of identifying such microtubule dependent mitotic kinesin modulating compounds, especially small organic molecules. The methods involve screening the “test” compound's ability to competitively inhibit binding of a moiety (e.g., ATP or an ATP analogue) at the ATPase site, screening the same compound's ability to competitively inhibit binding of a moiety (e.g., a microtubule) at the microtubule binding, and finally testing the activity of the kinesin motor protein after treatment with the test compound in the presence of microtubules and testing the activity of the kinesin motor protein after treatment with the test compound in the absence of microtubules and comparing those two activities.

Thus, in an embodiment, the instant method of identifying a compound that specifically modulates the activity of a kinesin motor protein bound to a microtubule, said kinesin motor protein having a microtubule binding site and a kinesin ATPase binding site, comprises the steps of:
a) assaying for competitive inhibition of said motor protein by said compound at said kinesin ATPase binding site;
b) assaying for competitive inhibition of said motor protein by said compound at said microtubule binding site;
c) assaying for inhibition of said motor protein by said compound in the absence of microtubules;
d) assaying for inhibition of said motor protein by said compound in the presence of microtubules;
e) identifying a compound as a kinesin-bound-to-microtubule modulator when said compound inhibits said motor protein activity in the presence of microtubules, is not a competitive modulator at said microtubule binding site and at said kinesin ATPase binding site, and does not inhibit said motor protein activity when microtubules are absent.

In an embodiment, the kinesin motor protein is KSP and the compound of the invention inhibits the kinesin motor activity of KSP.

Mitotic kinesin inhibitor compounds that are identified through the method described hereinabove may offer advantages over mitotic kinesin inhibitors that have been previously described in that the compounds identified through the instant method may be efficacious against tumors or cancers that are resistant to the mitotic kinesin inhibitors (previously described) that do not depend on the presence of microtubules for activity, but are instead competitive inhibitors with either microtubules or ATP.

Methods of identifying competitive inhibition are well known to those of skill in the art. Briefly, in the classical Michaelis-Menton model of enzyme kinetics, competitive inhibition is easily recognized experimentally because the percent inhibition at a fixed inhibitor concentration is decreased by increasing the substrate concentration. Thus, where a compound competitively inhibits binding at the microtubule binding site, increasing the microtubule concentration at a fixed concentration of test compound can restore the original (inhibitor-free) maximal rate of reaction (V max). Conversely, where inhibition is non-competitive, increasing the substrate concentration will not restore the maximal rate of reaction (Vmax). Assays for specific inhibition at the microtubule binding site are illustrated in example 1. For a detailed discussion of analysis of reaction kinetics to recognize competitive, noncompetitive and uncompetitive inhibition, see, e.g., Lehninger (1975) Biochemistry Worth Pub., Inc. New York, N.Y.

In another embodiment, this invention provides methods of modulating (e.g. inhibiting) kinesin motor activity in a cell. The methods involve contacting the cell with one of the compounds that have been identified as inhibiting a kinesin motor protein by the mechanism described above. The cell, although preferably a mammalian cell, need not be so limited. Other suitable cells include, but are not limited to, fungal cells and microbial cells. The cell can be in vitro or in vivo. Where the method is practiced in a therapeutic context (e.g. to ameliorate the effects of a pathological condition characterized by hyperproliferation of one or more cells) the compounds identified by the assays described herein as modulating KSP mitotic kinesin activity only when the kinesin is bound to a microtubule, while not acting as competitive inhibitors for microtubule binding and for ATP binding.

The compounds that have been identified as modulating KSP mitotic kinesin activity only when the kinesin is bound to a microtubule, while not acting as competitive inhibitors for microtubule binding and for ATP binding include:

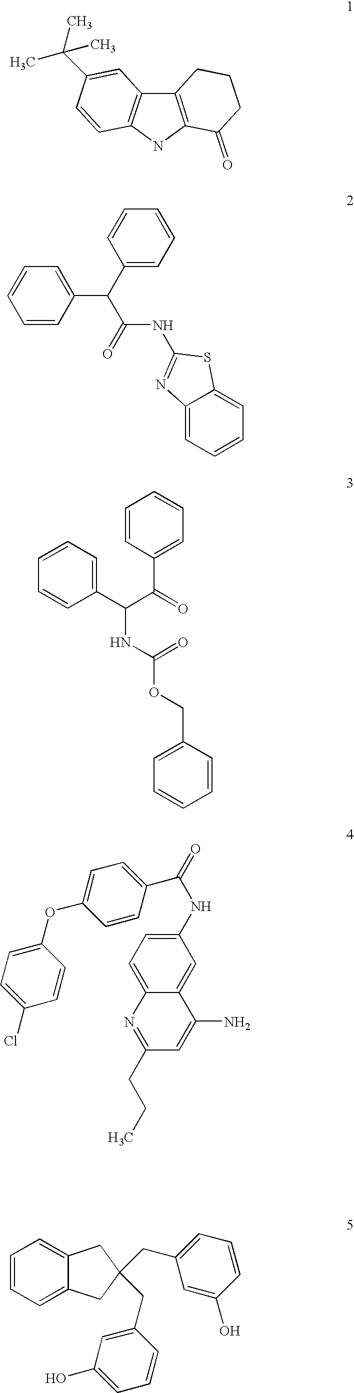

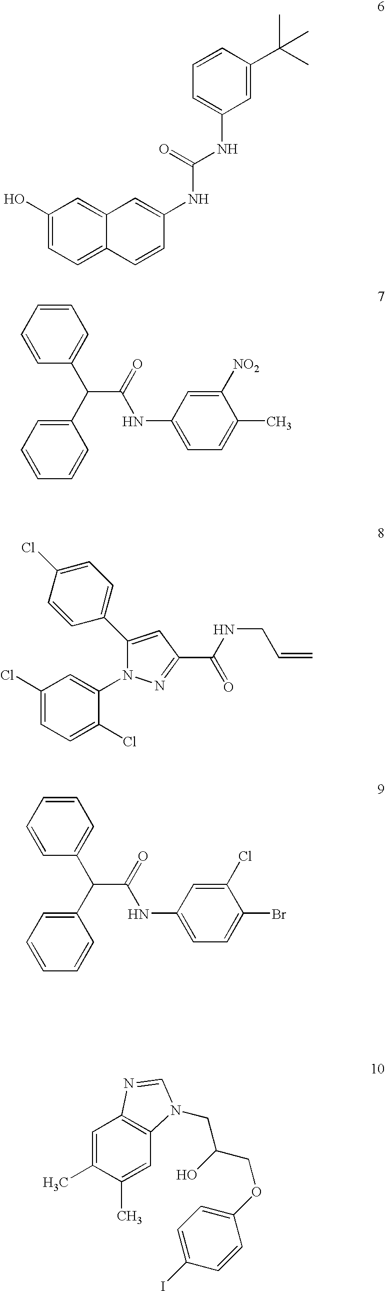

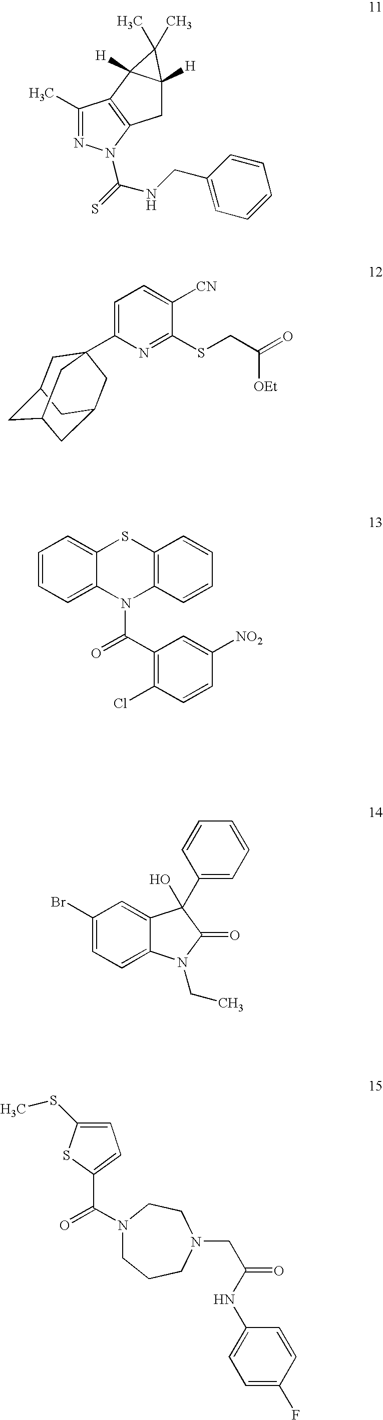

The term “molecular motor protein” refers to cytoskeletal molecule(s) that utilize chemical energy to produce mechanical force, and drive the motile properties of the cytoskeleton.

The terms “kinesin” and “kinesin superfamily” as used herein refer to a superfamily of eucaryotic motor proteins used to transport a large variety of cargoes along microtubule “tracks”. Members of the kinesin superfamily are believed to be essential for mitotic and meiotic spindle organization, chromosome segregation, organelle and vesicle transport and many other processes that require microtubule based transport. The common feature of kinesins in the presence of a conserved ˜350 amino acid motor domain which harbors the microtubule binding, ATP-hydrolyzing, and force transducing activities (see, e.g., Barton et al. (1996) Proc. Natl. Acad. Sci. USA, 93(5): 1735-1742, and Goldstein, (1993) Annu. Rev. Genet., 27: 319-351).

The term “kinesin motor protein” is used to refer to one or more proteins involved in the transduction of chemical energy into mechanical energy. Kinesin is a force generating enzyme that hydrolyzes ATP to ADP and Pᵢ and uses the derived chemical energy to induce protein movement; for example, plus end directed movement along microtubules. Not all kinesins induce plus end directed movement: some are minus end and some depolymerize microtubules. KSP is a plus-end kinesin. This ubiquitous microtubule motor is thought to power anterograde organelle transport along microtubules.

The term kinesin motor is intended to include kinesin related proteins inhibition of which inhibits kinesin motor activity. Kinesin heavy and light chains have been cloned and sequenced from a number of species including, but not limited to Drosophila (GenBank M24441), squid optic lobe (GenBank J05258), sea urchin and human (GenBank X65873), and rat (M75146, M75147, M75148), and the like (see, e.g., Yang et al. (1989) Cell 56: 879-889, Wright et al. (1991) J. Cell. Biol., 113: 817-833, Navone et al. (1992) J. Cell. Biol., 117:1263-1275, and Cyr et al. (1991) Proc. Natl. Acad. Sci. USA, 88: 10114-10118). In addition, the scientific literature is replete with detailed descriptions of kinesins (kinesin motors) and kinesin related proteins (see, e.g., Kreis and Vale (1993) Guidebook to the Cytoskeletal and Motor Proteins, Oxford University Press, Oxford, Vale (1990) Curr. Opin. Cell. Biol, 2: 15-22; Vale (1987) Ann. rev. Cell. Biol., 3: 347-378; and references therein).

The terms “kinesin motor inhibitor” or “inhibition of kinesin motor activity” refers to the decrease or elimination of kinesin/microtubule mediated transduction of chemical energy (e.g. as stored in ATP) into mechanical energy (e.g., force generation or movement). Such a decrease can be measured directly, e.g., as in a motility assay, or alternatively can be ascertained by the use of surrogate markers such as a decrease in the ATPase activity of the kinesin protein, and/or a decrease in the affinity and/or specificity of kinesin motor protein-microtubule binding interactions, and/or in a decrease in mitotic activity of a cell or cells. Conversely, a “kinesin motor agonist” or “upregulator of kinesin motor activity” refers to the increase of kinesin/microtubule mediated transduction of chemical energy (e.g. as stored in ATP) into mechanical energy (e.g. force generation or movement).

The term “test compound” refers to a compound whose anti-kinesin motor activity it is desired to determine. Such test compounds may include virtually any molecule or mixture of molecules, alone or in a suitable carrier.

The term “detecting the binding” means assessing the amount of a given second component that binds to a given first component in the presence and absence of a test composition. This process generally involves the ability to assess the amount of the second component associated with a known fixed amount of the first component at selected intervals after contacting the first and second components. This may be accomplished e.g., by attaching to the second component a molecule or functional group that can be visualized or measured (e.g., a fluorescent moiety, a radioactive atom, a biotin that can be detected using labeled avidin) or by using ligands that specifically bind to the second component. The level of binding is preferably detected quantitatively. Binding or a change in binding is indicated at the first detectable level. A change in binding, which can be an increase or a decrease, or presence versus absence, is preferably a change of at least about 10%, more preferably by at least about 20%, still more preferably by at least about 50%, still even more preferably by at least about 75%, even more preferably by at least about 150% or 200% and most preferably is a change of at least about 2 to about 10 fold (e.g., as compared to a control).

The phrase “detecting a change in kinesin motor activity resulting from said contacting” refers to determining the presence, absence or quantifying the alteration in kinesin motor activity caused by a particular composition (e.g. a test compound). The detecting can involve any one or more of a variety of assays for kinesin motor activity as described herein. A change in activity, which can be an increase or a decrease, or presence versus absence, is preferably a change of at least about 10%, more preferably by at least about 20%, still more preferably by at least about 50%, still even more preferably by at least about 75%, even more preferably by at least about 150% or 200% and most preferably is a change of at least about 2 to about 10 fold (e.g., as compared to a control).

The term “compound” as used herein refers to organic or inorganic molecules. The term includes, but is not limited to polypeptides, proteins, glycoproteins (e.g. antibodies), nucleic acids, oligonucleotides, and inorganic molecules.

The term “small organic molecule”, as used herein, refers to a compound that is an organic molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

By “protein” herein is meant at least two covalently attached amino acids, which includes proteins, polypeptides, oligopeptides, and peptides. The protein may be made of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline, and norleucine are considered amino acids for the purposes of this invention. “Amino acid” also includes imino acid residues such as proline and hydroxyproline. The side chains may be in either the (R) or the (S) configuration. In the preferred embodiment, the amino acids are in the (S) or L-configuration. If non-naturally occurring side chains are used, non-amino acid substituents may be used, for example to prevent or retard in vivo degradations.

The term “competitive inhibition” is used to refer to competitive inhibition in accord with the Michaelis-Menton model of enzyme kinetics. Competitive inhibition is recognized experimentally because the percent inhibition at a fixed inhibitor concentration is decreased by increasing the substrate concentration. At sufficiently high substrate concentration, V max can essentially be restored even in the presence of the inhibitor. Conversely, “non-competitive inhibition” refers to inhibition that is not reversed by increasing the substrate concentration.

The term “cell” is used to refer to any cell including, but not limited to mammalian, fungal, microbial and invertebrate cells. Preferred cells include tumor cells including, but not limited to, carcinomas, including breast, ovary, prostate, skin, and colon; brain cancers, including meningioma, glioma, oligodendroglioma, embryonic cancers; sarcomas; leukemias, and lymphomas. Preferred cells also include neurons. Particularly preferred neurons are those related to neurodegenerative diseases including Alzheimer's Disease, Parkinson's Disease, Huntington's Disease, Frontotemporal Dementias, and Amyotrophic Lateral Sclerosis. Preferred cells further include cells derived from the gastrointestinal system including esophagus, stomach, intestine, pancreas, liver, lung, heart, and vascular system as sell as cells from the central and peripheral nervous system, kidney, bladder, muscular system and the bone system.

“In vivo” refers to in the living body of an organism.

“In vitro” refers to outside the living body, such as, an artificial environment, for example, a test tube or a cell or tissue culture.

The term “modulate” as used herein refers to increasing or decreasing an activity of a molecule. Thus, for example, a kinesin motor modulator acts to increase or decrease (inhibit) kinesin motor activity.

### Utilities

The compounds identified by the methods of the invention find use in a variety of applications. As will be appreciated by those skilled in the art, mitosis may be altered in a variety of ways; that is, one can affect mitosis either by increasing or decreasing the activity of a component in the mitotic pathway. Stated differently, mitosis may be affected (e.g., disrupted) by disturbing equilibrium, either by inhibiting or activating certain components. Similar approaches may be used to alter meiosis.

The kinesin motor protein modulators of this invention are useful in a wide variety of contexts. In particular, preferred modulators of this invention act to inhibit activity of kinesin mediated transport. The kinesins (members of the kinesin superfamily) are implicated in microtubule-mediated transport activities. As such they participate in a wide variety of activities including, but not limited to mitotic and meiotic spindle organization, chromosome segregation, organelle and vesicle transport and many others processes that require microtubule based transport.

Modulation (e.g. inhibition) of kinesin motor proteins therefor has profound effect on cellular function acting, for example, to inhibit meiosis and/or mitosis, and consequently inhibiting cellular growth and/or proliferation, e.g. in vitro or in humans and other non-human animals. As powerful anti-mitotics or anti-meiotics, the kinesin inhibitors of this invention have a wide variety of uses, particularly in the treatment (e.g., amelioration) of, e.g. human and veterinary, pathological conditions characterized by abnormal cell proliferation. Such conditions include, but are not limited to: fungal infections, abnormal stimulation of endothelial cells (e.g., atherosclerosis), solid tumors and tumor metastasis, benign tumors, for example, hemangiomas, acoustic neuromas, neurofibromas, trachomas, and pyogenic granulomas, vascular malfunctions (e.g., arteria-venous malformations), abnormal wound healing, inflammatory and immune disorders, Bechet's disease, gout or gouty arthritis, abnormal angiogenesis accompanying: rheumatoid arthritis, psoriasis, diabetic retinopathy, and other ocular angiogenic diseases such as retinopathy of prematurity (retrolental fibroplastic), macular degeneration, corneal overgrowth, corneal graft rejection, neuroscular glaucoma, Oster Webber syndrome, and the like. In addition, it is expected the kinesin motor protein inhibitors of this invention are useful in the treatment/mitigation of a number of neurodegenerative disorders.

In an embodiment, the compounds of the invention are used to modulate mitotic spindle formation, thus causing prolonged cell cycle arrest in mitosis. By “modulate” herein is meant altering mitotic spindle formation, including increasing and decreasing spindle formation. By “mitotic spindle formation” herein is meant organization of microtubules into bipolar structures by mitotic kinesins. By “mitotic spindle dysfunction” herein is meant mitotic arrest and monopolar spindle formation.

The compounds of the invention are useful to bind to and/or modulate the activity of a mitotic kinesin. In an embodiment, the mitotic kinesin is a member of the bimC subfamily of mitotic kinesins (as described in U.S. Pat. No. 6,284,480, column 5). In a further embodiment, the mitotic kinesin is human KSP, although the activity of mitotic kinesins from other organisms may also be modulated by the compounds of the present invention. In this context, modulate means either increasing or decreasing spindle pole separation, causing malformation, i.e., splaying, of mitotic spindle poles, or otherwise causing morphological perturbation of the mitotic spindle. Also included within the definition of KSP for these purposes are variants and/or fragments of KSP. In addition, other mitotic kinesins may be inhibited by the compounds of the present invention.

The compounds of the invention are used to treat cellular proliferation diseases. Disease states which can be treated by the methods and compositions provided herein include, but are not limited to, cancer (further discussed below), autoimmune disease, arthritis, graft rejection, inflammatory bowel disease, proliferation induced after medical procedures, including, but not limited to, surgery, angioplasty, and the like. It is appreciated that in some cases the cells may not be in a hyper- or hypoproliferation state (abnormal state) and still require treatment. For example, during wound healing, the cells may be proliferating “normally”, but proliferation enhancement may be desired.

The compounds, compositions and methods provided herein are particularly deemed useful for the treatment of cancer including solid tumors such as skin, breast, brain, cervical carcinomas, testicular carcinomas, etc. In particular, cancers that may be treated by the compounds, compositions and methods of the invention include, but are not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chondroma, osteochondroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma. Thus, the term “cancerous cell” as provided herein, includes a cell afflicted by any one of the above-identified conditions.

The compounds of the instant invention may also be useful as antifungal agents, by modulating the activity of the fungal members of the bimC kinesin subgroup, as is described in U.S. Pat. No. 6,284,480.

The compounds of this invention may be administered to mammals, preferably humans, either alone or in combination with pharmaceutically acceptable carriers, excipients or diluents, in a pharmaceutical composition, according to standard pharmaceutical practice. The compounds can be administered orally or parenterally, including the intravenous, intramuscular, intraperitoneal, subcutaneous, rectal and topical routes of administration.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, microcrystalline cellulose, sodium crosscarmellose, corn starch, or alginic acid; binding agents, for example starch, gelatin, polyvinyl-pyrrolidone or acacia, and lubricating agents, for example, magnesium stearate, stearic acid or talc. The tablets may be uncoated or they may be coated by known techniques to mask the unpleasant taste of the drug or delay disintegration and absorption in the gastrointestinal tract and thereby provide a sustained action over a longer period. For example, a water soluble taste masking material such as hydroxypropyl-methylcellulose or hydroxypropylcellulose, or a time delay material such as ethyl cellulose, cellulose acetate butyrate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

Aqueous suspensions contain the active material in a mixture with excipients suitable for the manufacture of aqueous suspensions. Such excipients are suspending agents, for example sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

The pharmaceutical compositions of the invention may also be in the form of an oil-in-water emulsion. The oily phase may be a vegetable oil, for example olive oil or arachis oil, or a mineral oil, for example liquid paraffin or mixtures of these. Suitable emulsifying agents may be naturally occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening, flavoring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

The pharmaceutical compositions may be in the form of a sterile injectable aqueous solution. Among the acceptable vehicles and solvents that may be employed are water, Ringer's solution and isotonic sodium chloride solution.

The sterile injectable preparation may also be a sterile injectable oil-in-water microemulsion where the active ingredient is dissolved in the oily phase. For example, the active ingredient may be first dissolved in a mixture of soybean oil and lecithin. The oil solution then introduced into a water and glycerol mixture and processed to form a microemulation.

The injectable solutions or microemulsions may be introduced into a patient's blood stream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump.

The pharmaceutical compositions may be in the form of a sterile injectable aqueous or oleagenous suspension for intramuscular and subcutaneous administration. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, for example as a solution in 1,3-butane diol. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compounds of Formula I may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing the compound of Formula I are employed. (For purposes of this application, topical application shall include mouth washes and gargles.)

The compounds for the present invention can be administered in intranasal form via topical use of suitable intranasal vehicles and delivery devices, or via transdermal routes, using those forms of transdermal skin patches well known to those of ordinary skill in the art. To be administered in the form of a transdermal delivery system, the dosage administration will, of course, be continuous rather than intermittent throughout the dosage regimen. Compounds of the present invention may also be delivered as a suppository employing bases such as cocoa butter, glycerinated gelatin, hydrogenated vegetable oils, mixtures of polyethylene glycols of various molecular weights and fatty acid esters of polyethylene glycol.

When a compound according to this invention is administered into a human subject, the daily dosage will normally be determined by the prescribing physician with the dosage generally varying according to the age, weight, sex and response of the individual patient, as well as the severity of the patient's symptoms.

In one exemplary application, a suitable amount of compound is administered to a mammal undergoing treatment for cancer. Administration occurs in an amount between about 0.1 mg/kg of body weight to about 60 mg/kg of body weight per day, preferably of between 0.5 mg/kg of body weight to about 40 mg/kg of body weight per day.

The instant compounds are also useful in combination with known therapeutic agents and anti-cancer agents. For example, instant compounds are useful in combination with known anti-cancer agents. Combinations of the presently disclosed compounds with other anti-cancer or chemotherapeutic agents are within the scope of the invention. Examples of such agents can be found in Cancer Principles and Practice of Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Such anti-cancer agents include, but are not limited to, the following: estrogen receptor modulators, androgen receptor modulators, retinoid receptor modulators, cytotoxic/cytostatic agents, antiproliferative agents, prenyl-protein transferase inhibitors, HMG-CoA reductase inhibitors and other angiogenesis inhibitors, inhibitors of cell proliferation and survival signaling, apoptosis inducing agents and agents that interfere with cell cycle checkpoints. The instant compounds are particularly useful when co-administered with radiation therapy.

In an embodiment, the instant compounds are also useful in combination with known anti-cancer agents including the following: estrogen receptor modulators, androgen receptor modulators, retinoid receptor modulators, cytotoxic agents, antiproliferative agents, prenyl-protein transferase inhibitors, HMG-CoA reductase inhibitors, BV protease inhibitors, reverse transcriptase inhibitors, and other angiogenesis inhibitors.

“Estrogen receptor modulators” refers to compounds that interfere with or inhibit the binding of estrogen to the receptor, regardless of mechanism. Examples of estrogen receptor modulators include, but are not limited to, tamoxifen, raloxifene, idoxifene, LY353381, LY117081, toremifene, fulvestrant, 4-[7-(2,2-dimethyl-1-oxopropoxy-4-methyl-2-[4-[2-(1-piperidinyl)ethoxy]phenyl]-2H-1-benzopyran-3-yl]-phenyl-2,2-dimethylpropanoate, 4,4′-dihydroxybenzophenone-2,4-dinitrophenyl-hydrazone, and SH646.

“Androgen receptor modulators” refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole, and abiraterone acetate.

“Retinoid receptor modulators” refers to compounds which interfere or inhibit the binding of retinoids to the receptor, regardless of mechanism. Examples of such retinoid receptor modulators include bexarotene, tretinoin, 13-cis-retinoic acid, 9-cis-retinoic acid, α-difluoromethylornithine, ILX23-7553, trans-N-(4′-hydroxyphenyl) retinamide, and N-4-carboxyphenyl retinamide.

“Cytotoxic/cytostatic agents” refer to compounds which cause cell death or inhibit cell proliferation primarily by interfering directly with the cell's functioning or inhibit or interfere with cell mitosis, including alkylating agents, tumor necrosis factors, intercalators, hypoxia activatable compounds, microtubule inhibitors/microtubule-stabilizing agents, inhibitors of mitotic kinesins, inhibitors of kinases involved in mitotic progression, antimetabolites; biological response modifiers; hormonal/anti-hormonal therapeutic agents, hematopoietic growth factors, monoclonal antibody targeted therapeutic agents, topoisomerase inhibitors, proteasome inhibitors and ubiquitin ligase inhibitors.

Examples of cytotoxic agents include, but are not limited to, sertenef, cachectin, ifosfamide, tasonermin, lonidamine, carboplatin, altretamine, prednimustine, dibromodulcitol, ranimustine, fotemustine, nedaplatin, oxaliplatin, temozolomide, heptaplatin, estramustine, improsulfan tosilate, trofosfamide, nimustine, dibrospidium chloride, pumitepa, lobaplatin, satraplatin, profiromycin, cisplatin, irofulven, dexifosfamide, cis-aminedichloro(2-methyl-pyridine)platinum, benzylguanine, glufosfamide, GPX100, (trans, trans, trans)-bis-mu-(hexane-1,6-diamine)-mu-[diamine-platinum(II)]bis[diamine(chloro)platinum (II)]tetrachloride, diarizidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-deamino-3′-morpholino-13-deoxo-10-hydroxycamminomycin, annamycin, galarubicin, elinafide, MEN10755, and 4-demethoxy-3-deamino-3-aziridinyl-4-methylsulphonyl-daunorubicin (see WO 00/50032).

An example of a hypoxia activatable compound is tirapazamine.

Examples of proteasome inhibitors include but are not limited to lactacystin and bortezomib.

Examples of microtubule inhibitors/microtubule-stabilising agents include paclitaxel, vindesine sulfate, 3′,4′-didehydro-4′-deoxy-8′-norvincaleukoblastine, docetaxol, rhizoxin, dolastatin, mivobulin isethionate, auristatin, cemadotin, RPR109881, BMS184476, vinflunine, cryptophycin, 2,3,4,5,6-pentafluoro-N-(3-fluoro-4-methoxyphenyl) benzene sulfonamide, anhydrovinblastine, N,N-dimethyl-L-valyl-L-valyl-N-methyl-L-valyl-L-prolyl-L-proline-t-butylamide, TDX258, the epothilones (see for example U.S. Pat. Nos. 6,284,781 and 6,288,237) and BMS188797.

Some examples of topoisomerase inhibitors are topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-k1]acridine-2-(6H) propanamine, 1-amino-9-ethyl-5-fluoro-2,3-dihydro-9-hydroxy-4-methyl-1H,12H-benzo[de]pyrano[3′,4′:b,7]-indolizino[1,2b]quinoline-10,13(9H, 15H)dione, lurtotecan, 7-[2-(N-isopropylamino)ethyl]-(20S)camptothecin, BNP1350, BNPI1100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a, 5aB, 8aa,9b)-9-[2-[N-[2-(dimethylamino)ethyl]-N-methylamino]ethyl]-5-[4-hydroOxy-3,5-dimethoxyphenyl]-5,5a,6,8,8a,9-hexohydrofuro(3′,4′:6,7)naphtho(2,3-d)-1,3-dioxol-6-one, 2,3-(methylenedioxy)-5-methyl-7-hydroxy-8-methoxybenzo[c]-phenanthridinium, 6,9-bis[(2-aminoethyl)amino]benzo[g]isoquinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2(diethylamino)ethylamino]-7-methoxy-9-oxo-9H-thioxanthen-4-ylmethyl]formamide, N-(2-(dimethylamino)ethyl)acridine-4-carboxamide, 6-[[2-(dimethylamino)ethyl]amino]-3-hydroxy-7H-indeno[2,1-c]quinolin-7-one, and dimesna.

Examples of inhibitors of mitotic kinesins, and in particular the human mitotic kinesin KSP, are described in PCT Publications WO 01/30768, WO 01/98278, WO 03/050,064, WO 03/050,122, WO 03/049,527, WO 03/049,679, WO 03/049,678 and WO 03/39460 and pending PCT Appl. Nos. US03/06403 (filed Mar. 4, 2003), US03/15861 (filed May 19, 2003), US03/15810 (filed May 19, 2003), US03/18482 (filed Jun. 12, 2003) and US03/18694 (filed Jun. 12, 2003). In an embodiment inhibitors of mitotic kinesins include, but are not limited to inhibitors of KSP, inhibitors of MKLP1, inhibitors of CENP-E, inhibitors of MCAK, inhibitors of Kif14, inhibitors of Mphosph1 and inhibitors of Rab6-KIFL.

“Inhibitors of kinases involved in mitotic progression” include, but are not limited to, inhibitors of aurora kinase, inhibitors of Polo-like kinases (PLK) (in particular inhibitors of PLK-1), inhibitors of bub-1 and inhibitors of bub-R1.

“Antiproliferative agents” includes antisense RNA and DNA oligonucleotides such as G3139, ODN698, RVASKRAS, GEM231, and INX3001, and antimetabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, 2′-fluoromethylene-2′-deoxycytidine, N-[5-(2,3-dihydro-benzofuryl)sulfonyl]-N′-(3,4-dichlorophenyl)urea, N6-[4-deoxy-4-[N2-[2(E),4(E)-tetradecadienoyl]glycylamino]-L-glycero-B-L-manno-heptopyranosyl]adenine, aplidine, ecteinascidin, troxacitabine, 4-[2-amino-4-oxo-4,6,7,8-tetrahydro-3H-pyrimidino[5,4-b][1,4]thiazin-6-yl-(S)-ethyl]-2,5-thienoyl-L-glutamic acid, aminopterin, 5-fluorouracil, alanosine, 11-acetyl-8-(carbamoyloxymethyl)-4-formyl-6-methoxy-14-oxa-1,11-diazatetracyclo(7.4.1.0.0)-tetradeca-2,4,6-trien-9-yl acetic acid ester, swainsonine, lometrexol, dexrazoxane, methioninase, 2′-cyano-2′-deoxy-N4-palmitoyl-1-B-D-arabino furanosyl cytosine and 3-aminopyridine-2-carboxaldehyde thiosemicarbazone.

Examples of monoclonal antibody targeted therapeutic agents include those therapeutic agents which have cytotoxic agents or radioisotopes attached to a cancer cell specific or target cell specific monoclonal antibody. Examples include Bexxar.

“HMG-CoA reductase inhibitors” refers to inhibitors of 3-hydroxy-3-methylglutaryl-CoA reductase. Examples of HMG-CoA reductase inhibitors that may be used include but are not limited to lovastatin (MEVACOR®; see U.S. Pat. Nos. 4,231,938, 4,294,926 and 4,319,039), simvastatin (ZOCOR®; see U.S. Pat. Nos. 4,444,784, 4,820,850 and 4,916,239), pravastatin (PRAVACHOL®; see U.S. Pat. Nos. 4,346,227, 4,537,859, 4,410,629, 5,030,447 and 5,180,589), fluvastatin (LESCOL®; see U.S. Pat. Nos. 5,354,772, 4,911,165, 4,929,437, 5,189,164, 5,118,853, 5,290,946 and 5,356,896) and atorvastatin (LIPITOR®; see U.S. Pat. Nos. 5,273,995, 4,681,893, 5,489,691 and 5,342,952). The structural formulas of these and additional HMG-CoA reductase inhibitors that may be used in the instant methods are described at page 87 of M. Yalpani, “Cholesterol Lowering Drugs”, Chemistry & Industry, pp. 85-89 (5 Feb. 1996) and U.S. Pat. Nos. 4,782,084 and 4,885,314. The term HMG-CoA reductase inhibitor as used herein includes all pharmaceutically acceptable lactone and open-acid forms (i.e., where the lactone ring is opened to form the free acid) as well as salt and ester forms of compounds which have HMG-CoA reductase inhibitory activity, and therefor the use of such salts, esters, open-acid and lactone forms is included within the scope of this invention.

“Prenyl-protein transferase inhibitor” refers to a compound which inhibits any one or any combination of the prenyl-protein transferase enzymes, including farnesyl-protein transferase (FPTase), geranylgeranyl-protein transferase type I (GGPTase-I), and geranylgeranyl-protein transferase type-II (GGPTase-II, also called Rab GGPTase).

Examples of prenyl-protein transferase inhibitors can be found in the following publications and patents: WO 96/30343, WO 97/18813, WO 97/21701, WO 97/23478, WO 97/38665, WO 98/28980, WO 98/29119, WO 95/32987, U.S. Pat. No. 5,420,245, U.S. Pat. No. 5,523,430, U.S. Pat. No. 5,532,359, U.S. Pat. No. 5,510,510, U.S. Pat. No. 5,589,485, U.S. Pat. No. 5,602,098, European Patent Publ. 0 618 221, European Patent Publ. 0 675 112, European Patent Publ. 0 604 181, European Patent Publ. 0 696 593, WO 94/19357, WO 95/08542, WO 95/11917, WO 95/12612, WO 95/12572, WO 95/10514, U.S. Pat. No. 5,661,152, WO 95/10515, WO 95/10516, WO 95/24612, WO 95/34535, WO 95/25086, WO 96/05529, WO 96/06138, WO 96/06193, WO 96/16443, WO 96/21701, WO 96/21456, WO 96/22278, WO 96/24611, WO 96/24612, WO 96/05168, WO 96/05169, WO 96/00736, U.S. Pat. No. 5,571,792, WO 96/17861, WO 96/33159, WO 96/34850, WO 96/34851, WO 96/30017, WO 96/30018, WO 96/30362, WO 96/30363, WO 96/31111, WO 96/31477, WO 96/31478, WO 96/31501, WO 97/00252, WO 97/03047, WO 97/03050, WO 97/04785, WO 97/02920, WO 97/17070, WO 97/23478, WO 97/26246, WO 97/30053, WO 97/44350, WO 98/02436, and U.S. Pat. No. 5,532,359.

For an example of the role of a prenyl-protein transferase inhibitor on angiogenesis see European J. of Cancer, Vol. 35, No. 9, pp. 1394-1401 (1999).

“Angiogenesis inhibitors” refers to compounds that inhibit the formation of new blood vessels, regardless of mechanism. Examples of angiogenesis inhibitors include, but are not limited to, tyrosine kinase inhibitors, such as inhibitors of the tyrosine kinase receptors Flt-1 (VEGFR1) and Flk-1/KDR (VEGFR2), inhibitors of epidermal-derived, fibroblast-derived, or platelet derived growth factors, MMP (matrix metalloprotease) inhibitors, integrin blockers, interferon-α, interleukin-12, pentosan polysulfate, cyclooxygenase inhibitors, including nonsteroidal anti-inflammatories (NSAIDs) like aspirin and ibuprofen as well as selective cyclooxy-genase-2 inhibitors like celecoxib and rofecoxib (PNAS, Vol. 89, p. 7384 (1992); JNCI, Vol. 69, p. 475 (1982); Arch. Opthalmol., Vol. 108, p. 573 (1990); Anat. Rec., Vol. 238, p. 68 (1994); FEBS Letters, Vol. 372, p. 83 (1995); Clin, Orthop. Vol. 313, p. 76 (1995); J. Mol. Endocrinol., Vol. 16, p. 107 (1996); Jpn. J. Pharmacol., Vol. 75, p. 105 (1997); Cancer Res., Vol. 57, p. 1625 (1997); Cell, Vol. 93, p. 705 (1998); Intl. J. Mol. Med., Vol. 2, p. 715 (1998); J. Biol. Chem., Vol. 274, p. 9116 (1999)), steroidal anti-inflammatories (such as corticosteroids, mineralocorticoids, dexamethasone, prednisone, prednisolone, methylpred, betamethasone), carboxyamidotriazole, combretastatin A-4, squalamine, 6-O-chloroacetyl-carbonyl)-fumagillol, thalidomide, angiostatin, troponin-1, angiotensin II antagonists (see Fernandez et al., J. Lab. Clin. Med. 105:141-145 (1985)), and antibodies to VEGF (see, Nature Biotechnology, Vol. 17, pp. 963-968 (October 1999); Kim et al., Nature, 362, 841-844 (1993); WO 00/44777; and WO 00/61186).

Other therapeutic agents that modulate or inhibit angiogenesis and may also be used in combination with the compounds of the instant invention include agents that modulate or inhibit the coagulation and fibrinolysis systems (see review in Clin. Chem. La. Med. 38:679-692 (2000)). Examples of such agents that modulate or inhibit the coagulation and fibrinolysis pathways include, but are not limited to, heparin (see Thromb. Haemost. 80:10-23 (1998)), low molecular weight heparins and carboxypeptidase U inhibitors (also known as inhibitors of active thrombin activatable fibrinolysis inhibitor [TAFIa]) (see Thrombosis Res. 101:329-354 (2001)). TAFIa inhibitors have been described in PCT Publication WO 03/013,526 and US, Ser. No. 60/349,925 (filed Jan. 18, 2002).

“Agents that interfere with cell cycle checkpoints” refer to compounds that inhibit protein kinases that transduce cell cycle checkpoint signals, thereby sensitizing the cancer cell to DNA damaging agents. Such agents include inhibitors of ATR, ATM, the Chk1 and Chk2 kinases and Cdk and Cdc kinase inhibitors and are specifically exemplified by 7-hydroxystaurosporin, flavopiridol, CYC202 (Cyclacel) and BMS-387032.

“Inhibitors of cell proliferation and survival signaling pathway” refer to pharmaceutical agents that inhibit cell surface receptors and signal transduction cascades downstream of those surface receptors. Such agents include inhibitors of inhibitors of EGFR (for example gefitinib and erlotinib), inhibitors of ERB-2 (for example trastuzumab), inhibitors of IGFR, inhibitors of cytokine receptors, inhibitors of MET, inhibitors of PI3K (for example LY294002), serine/threonine kinases (including but not limited to inhibitors of Akt such as described in WO 02/083064, WO 02/083139, WO 02/083140 and WO 02/083138), inhibitors of Raf kinase (for example BAY-43-9006), inhibitors of MBK (for example CI-1040 and PD-098059) and inhibitors of mTOR (for example Wyeth CCI-779). Such agents include small molecule inhibitor compounds and antibody antagonists.

“Apoptosis inducing agents” include activators of TNF receptor family members (including the TRAIL receptors).

The invention also encompasses combinations with NSAID's which are selective COX-2 inhibitors. For purposes of this specification NSAID's which are selective inhibitors of COX-2 are defined as those which possess a specificity for inhibiting COX-2 over COX-1 of at least 100 fold as measured by the ratio of IC₅₀ for COX-2 over IC₅₀ for COX-1 evaluated by cell or microsomal assays. Such compounds include, but are not limited to those disclosed in U.S. Pat. No. 5,474,995, U.S. Pat. No. 5,861,419, U.S. Pat. No. 6,001,843, U.S. Pat. No. 6,020,343, U.S. Pat. No. 5,409,944, U.S. Pat. No. 5,436,265, U.S. Pat. No. 5,536,752, U.S. Pat. No. 5,550,142, U.S. Pat. No. 5,604,260, U.S. Pat. No. 5,698,584, U.S. Pat. No. 5,710,140, WO 94/15932, U.S. Pat. No. 5,344,991, U.S. Pat. No. 5,134,142, U.S. Pat. No. 5,380,738, U.S. Pat. No. 5,393,790, U.S. Pat. No. 5,466,823, U.S. Pat. No. 5,633,272, and U.S. Pat. No. 5,932,598, all of which are hereby incorporated by reference.

Inhibitors of COX-2 that are particularly useful in the instant method of treatment are: 3-phenyl-4-(4-(methylsulfonyl)phenyl)-2-(5H)-furanone; and 5-chloro-3-(4-methylsulfonyl)phenyl-2-(2-methyl-5-pyridinyl)pyridine;

or a pharmaceutically acceptable salt thereof.

Compounds that have been described as specific inhibitors of COX-2 and are therefore useful in the present invention include, but are not limited to: parecoxib, CELEBREX® and BEXTRA® or a pharmaceutically acceptable salt thereof.

Other examples of angiogenesis inhibitors include, but are not limited to, endostatin, ukrain, ranpirnase, IM862, 5-methoxy-4-[2-methyl-3-(3-methyl-2-butenyl)oxiranyl]-1-oxaspiro[2,5]oct-6-yl(chloroacetyl)carbamate, acetyldinanaline, 5-amino-1-[[3,5-dichloro-4-(4-chlorobenzoyl)phenyl]methyl]-1H-1,2,3-triazole-4-carboxamide, CM101, squalamine, combretastatin, RPI4610, NX31838, sulfated mannopentaose phosphate, 7,7-(carbonyl-bis[imino-N-methyl-4,2-pyrrolocarbonylimino[N-methyl-4,2-pyrrole]-carbonylimino]-bis-(1,3-naphthalene disulfonate), and 3-[(2,4-dimethylpyrrol-5-yl)methylene]-2-indolinone (SU5416).

As used above, “integrin blockers” refers to compounds which selectively antagonize, inhibit or counteract binding of a physiological ligand to the αᵥβ₃ integrin, to compounds which selectively antagonize, inhibit or counteract binding of a physiological ligand to the αvβ5 integrin, to compounds which antagonize, inhibit or counteract binding of a physiological ligand to both the αᵥβ₃ integrin and the αᵥβ₅ integrin, and to compounds which antagonize, inhibit or counteract the activity of the particular integrin(s) expressed on capillary endothelial cells. The term also refers to antagonists of the αᵥβ₆, αᵥβ₈, α₁β₁, α₂β₁, α₅β₁, α₆β₁ and α₆β₄ integrins. The term also refers to antagonists of any combination of αᵥβ₃, αᵥβ₅, αᵥβ₆, αᵥβ₈, α₁β₁, α₂β₁, α₅β₁, α₆β₁ and α₆β₄ integrins.

Some specific examples of tyrosine kinase inhibitors include N-(trifluoromethylphenyl)-5-methylisoxazol-4-carboxamide, 3-[(2,4-dimethylpyrrol-5-yl)methylidenyl)indolin-2-one, 17-(allylamino)-17-demethoxygeldanamycin, 4-(3-chloro-4-fluorophenylamino)-7-methoxy-6-[3-(4-morpholinyl)propoxyl]quinazoline, N-(3-ethynylphenyl)-6,7-bis(2-methoxyethoxy)-4-quinazolinamine, BIBX1382, 2,3,9,10,11,12-hexahydro-10-(hydroxymethyl)-10-hydroxy-9-methyl-9,12-epoxy-1H-diindolo[1,2,3-fg:3′,2′,1′-k1]pyrrolo[3,4-i][1,6]benzodiazocin-1-one, SH268, genistein, STI571, CEP2563, 4-(3-chlorophenylamino)-5,6-dimethyl-7H-pyrrolo[2,3-d]pyrimidinemethane sulfonate, 4-(3-bromo-4-hydroxyphenyl)amino-6,7-dimethoxyquinazoline, 4-(4′-hydroxyphenyl)amino-6,7-dimethoxyquinazoline, SU6668, STI571A, N-4-chlorophenyl-4-(4-pyridylmethyl)-1-phthalazinamine, and EMD121974.

Combinations with compounds other than anti-cancer compounds are also encompassed in the instant methods. For example, combinations of the instantly claimed compounds with PPAR-γ (i.e., PPAR-gamma) agonists and PPAR-δ (i.e., PPAR-delta) agonists are useful in the treatment of certain malignancies. PPAR-γ and PPAR-δ are the nuclear peroxisome proliferator-activated receptors γ and δ. The expression of PPAR-γ on endothelial cells and its involvement in angiogenesis has been reported in the literature (see J. Cardiovasc. Pharmacol. 1998; 31:909-913; J. Biol. Chem. 1999; 274:9116-9121; Invest. Ophthalmol Vis. Sci. 2000; 41:2309-2317). More recently, PPAR-γ agonists have been shown to inhibit the angiogenic response to VEGF in vitro; both troglitazone and rosiglitazone maleate inhibit the development of retinal neovascularization in mice. (Arch. Ophthamol. 2001; 119:709-717). Examples of PPAR-γ agonists and PPAR-γ/α agonists include, but are not limited to, thiazolidinediones (such as DRF2725, CS-011, troglitazone, rosiglitazone, and pioglitazone), fenofibrate, gemfibrozil, clofibrate, GW2570, SB219994, AR-H039242, JTT-501, MCC-555, GW2331, GW409544, NN2344, KRP297, NP0110, DRF4158, NN622, G1262570, PNU182716, DRF552926, 2-[(5,7-dipropyl-3-trifluoromethyl-1,2-benzisoxazol-6-yl)oxy]-2-methylpropionic acid (disclosed in U.S. Ser. No. 09/782,856), and 2(R)-7-(3-(2-chloro-4-(4-fluorophenoxy)phenoxy)propoxy)-2-ethylchromane-2-carboxylic acid (disclosed in U.S. Ser. Nos. 60/235,708 and 60/244,697).

Another embodiment of the instant invention is the use of the presently disclosed compounds in combination with gene therapy for the treatment of cancer. For an overview of genetic strategies to treating cancer see Hall et al (Am J Hum Genet. 61:785-789, 1997) and Kufe et al (Cancer Medicine, 5th Ed, pp 876-889, BC Decker, Hamilton 2000). Gene therapy can be used to deliver any tumor suppressing gene. Examples of such genes include, but are not limited to, p53, which can be delivered via recombinant virus-mediated gene transfer (see U.S. Pat. No. 6,069,134, for example), a uPA/uPAR antagonist (“Adenovirus-Mediated Delivery of a uPA/uPAR Antagonist Suppresses Angiogenesis-Dependent Tumor Growth and Dissemination in Mice,” Gene Therapy, August 1998; 5(8):1105-13), and interferon gamma (J Immunol 2000; 164:217-222).

The compounds of the instant invention may also be administered in combination with an inhibitor of inherent multidrug resistance (MDR), in particular MDR associated with high levels of expression of transporter proteins. Such MDR inhibitors include inhibitors of p-glycoprotein (P-gp), such as LY335979, XR9576, OC144-093, R101922, VX853 and PSC833 (valspodar).

A compound of the present invention may be employed in conjunction with anti-emetic agents to treat nausea or emesis, including acute, delayed, late-phase, and anticipatory emesis, which may result from the use of a compound of the present invention, alone or with radiation therapy. For the prevention or treatment of emesis, a compound of the present invention may be used in conjunction with other anti-emetic agents, especially neurokinin-1 receptor antagonists, 5HT3 receptor antagonists, such as ondansetron, granisetron, tropisetron, and zatisetron, GABAB receptor agonists, such as baclofen, a corticosteroid such as Decadron (dexamethasone), Kenalog, Aristocort, Nasalide, Preferid, Benecorten or others such as disclosed in U.S. Pat. Nos. 2,789,118, 2,990,401, 3,048,581, 3,126,375, 3,929,768, 3,996,359, 3,928,326 and 3,749,712, an antidopaminergic, such as the phenothiazines (for example prochlorperazine, fluphenazine, thioridazine and mesoridazine), metoclopramide or dronabinol. In an embodiment, an anti-emesis agent selected from a neurokinin-1 receptor antagonist, a 5HT3 receptor antagonist and a corticosteroid is administered as an adjuvant for the treatment or prevention of emesis that may result upon administration of the instant compounds.

Neurokinin-1 receptor antagonists of use in conjunction with the compounds of the present invention are fully described, for example, in U.S. Pat. Nos. 5,162,339, 5,232,929, 5,242,930, 5,373,003, 5,387,595, 5,459,270, 5,494,926, 5,496,833, 5,637,699, 5,719,147; European Patent Publication Nos. EP 0 360 390, 0 394 989, 0 428 434, 0 429 366, 0 430 771, 0 436 334, 0 443 132, 0 482 539, 0 498 069, 0499 313, 0 512 901, 0 512 902, 0 514 273, 0 514 274, 0 514 275, 0 514 276, 0 515 681, 0 517 589, 0 520 555, 0 522 808, 0 528 495, 0 532 456, 0 533 280, 0 536 817, 0 545 478, 0 558 156, 0 577 394, 0 585 913, 0 590 152, 0 599 538, 0 610 793, 0 634 402, 0 686 629, 0 693 489, 0 694 535, 0 699 655, 0 699 674, 0 707 006, 0 708 101, 0 709 375, 0 709 376, 0 714 891, 0 723 959, 0 733 632 and 0 776 893; PCT International Patent Publication Nos. WO 90/05525, 90/05729, 91/09844, 91/18899, 92/01688, 92/06079, 92/12151, 92/15585, 92/17449, 92/20661, 92/20676, 92/21677, 92/22569, 93/00330, 93/00331, 93/01159, 93/01165, 93/01169, 93/01170, 93/06099, 93/09116, 93/10073, 93/14084, 93/14113, 93/18023, 93/19064, 93/21155, 93/21181, 93/23380, 93/24465, 94/00440, 94/01402, 94/02461, 94/02595, 94/03429, 94/03445, 94/04494, 94/04496, 94/05625, 94/07843, 94/08997, 94/10165, 94/10167, 94/10168, 94/10170, 94/11368, 94/13639, 94/13663, 94/14767, 94/15903, 94/19320, 94/19323, 94/20500, 94/26735, 94/26740, 94/29309, 95/02595, 95/04040, 95/04042, 95/06645, 95/07886, 95/07908, 95/08549, 95/11880, 95/14017, 95/15311, 95/16679, 95/17382, 95/18124, 95/18129, 95/19344, 95/20575, 95/21819, 95/22525, 95/23798, 95/26338, 95/28418, 95/30674, 95/30687, 95/33744, 96/05181, 96/05193, 96/05203, 96/06094, 96/07649, 96/10562, 96/16939, 96/18643, 96/20197, 96/21661, 96/29304, 96/29317, 96/29326, 96/29328, 96/31214, 96/32385, 96/37489, 97/01553, 97/01554, 97/03066, 97/08144, 97/14671, 97/17362, 97/18206, 97/19084, 97/19942 and 97/21702; and in British Patent Publication Nos. 2 266 529, 2 268 931, 2 269 170, 2 269 590, 2 271 774, 2 292 144, 2 293 168, 2 293 169, and 2 302 689. The preparation of such compounds is fully described in the aforementioned patents and publications, which are incorporated herein by reference.

In an embodiment, the neurokinin-1 receptor antagonist for use in conjunction with the compounds of the present invention is selected from: 2-(R)-(1-(R)-(3,5-bis(trifluoromethyl)phenyl)ethoxy)-3-(S)-(4-fluorophenyl)-4-(3-(5-oxo-1H,4H-1,2,4-triazolo)methyl)morpholine, or a pharmaceutically acceptable salt thereof, which is described in U.S. Pat. No. 5,719,147.

A compound of the instant invention may also be administered with an agent useful in the treatment of anemia. Such an anemia treatment agent is, for example, a continuous erythropoiesis receptor activator (such as epoetin alfa).

A compound of the instant invention may also be administered with an agent useful in the treatment of neutropenia. Such a neutropenia treatment agent is, for example, a hematopoietic growth factor which regulates the production and function of neutrophils such as a human granulocyte colony stimulating factor, (G-CSF). Examples of a G-CSF include filgrastim.

A compound of the instant invention may also be administered with an immunologic-enhancing drug, such as levamisole, isoprinosine and Zadaxin.

Thus, the scope of the instant invention encompasses the use of the instantly claimed compounds in combination with a second compound selected from: an estrogen receptor modulator, an androgen receptor modulator, retinoid receptor modulator, a cytotoxic/cytostatic agent, an antiproliferative agent, a prenyl-protein transferase inhibitor, an HMG-CoA reductase inhibitor, an HIV protease inhibitor, a reverse transcriptase inhibitor, an angiogenesis inhibitor, a PPAR-γ agonist, a PPAR-δ agonist, an inhibitor of inherent multidrug resistance, an anti-emetic agent, an agent useful in the treatment of anemia, an agent useful in the treatment of neutropenia, an immunologic-enhancing drug, an inhibitor of cell proliferation and survival signaling, an agent that interferes with a cell cycle checkpoint, and an apoptosis inducing agent.

The term “administration” and variants thereof (e.g., “administering” a compound) in reference to a compound of the invention means introducing the compound or a prodrug of the compound into the system of the animal in need of treatment. When a compound of the invention or prodrug thereof is provided in combination with one or more other active agents (e.g., a cytotoxic agent, etc.), “administration” and its variants are each understood to include concurrent and sequential introduction of the compound or prodrug thereof and other agents.

As used herein, the term “composition” is intended to encompass a product comprising the specified ingredients in the specified amounts, as well as any product which results, directly or indirectly, from combination of the specified ingredients in the specified amounts.

The term “therapeutically effective amount” as used herein means that amount of active compound or pharmaceutical agent that elicits the biological or medicinal response in a tissue, system, animal or human that is being sought by a researcher, veterinarian, medical doctor or other clinician.

The term “treating cancer” or “treatment of cancer” refers to administration to a mammal afflicted with a cancerous condition and refers to an effect that alleviates the cancerous condition by killing the cancerous cells, but also to an effect that results in the inhibition of growth and/or metastasis of the cancer.

In an embodiment, the angiogenesis inhibitor to be used as the second compound is selected from a tyrosine kinase inhibitor, an inhibitor of epidermal-derived growth factor, an inhibitor of fibroblast-derived growth factor, an inhibitor of platelet derived growth factor, an MMP (matrix metalloprotease) inhibitor, an integrin blocker, interferon-α, interleukin-12, pentosan polysulfate, a cyclooxygenase inhibitor, carboxyamidotriazole, combretastatin A-4, squalamine, 6-O-chloroacetyl-carbonyl)-fumagillol, thalidomide, angiostatin, troponin-1, or an antibody to VEGF. In an embodiment, the estrogen receptor modulator is tamoxifen or raloxifene.

Also included in the scope of the claims is a method of treating cancer that comprises administering a therapeutically effective amount of a compound of Formula I in combination with radiation therapy and/or in combination with a compound selected from: an estrogen receptor modulator, an androgen receptor modulator, retinoid receptor modulator, a cytotoxic/cytostatic agent, an antiproliferative agent, a prenyl-protein transferase inhibitor, an HMG-CoA reductase inhibitor, an HIV protease inhibitor, a reverse transcriptase inhibitor, an angiogenesis inhibitor, a PPAR-γ agonist, a PPAR-δ agonist, an inhibitor of inherent multidrug resistance, an anti-emetic agent, an agent useful in the treatment of anemia, an agent useful in the treatment of neutropenia, an immunologic-enhancing drug, an inhibitor of cell proliferation and survival signaling, an agent that interferes with a cell cycle checkpoint, and an apoptosis inducing agent.

And yet another embodiment of the invention is a method of treating cancer that comprises administering a therapeutically effective amount of a compound of Formula I in combination with paclitaxel or trastuzumab.

The invention further encompasses a method of treating or preventing cancer that comprises administering a therapeutically effective amount of a compound of Formula I in combination with a COX-2 inhibitor.

The instant invention also includes a pharmaceutical composition useful for treating or preventing cancer that comprises a therapeutically effective amount of a compound of Formula I and a compound selected from: an estrogen receptor modulator, an androgen receptor modulator, a retinoid receptor modulator, a cytotoxic/cytostatic agent, an antiproliferative agent, a prenyl-protein transferase inhibitor, an HMG-CoA reductase inhibitor, an HIV protease inhibitor, a reverse transcriptase inhibitor, an angiogenesis inhibitor, a PPAR-γ agonist, a PPAR-δ agonist; an inhibitor of cell proliferation and survival signaling, an agent that interferes with a cell cycle checkpoint, and an apoptosis inducing agent.

### ATPase Assay

Kinesin motors are effective ATPases hydrolyzing ATP to ADP, thereby providing energy for force generation. By examining ADP release from kinesin in the presence of varying concentrations of kinesin motor modulator (e.g., adociasulfate), the activity of the kinesin motor modulator can be quantified. One such ADP release assay is described in the examples. In another embodiment, the ATPase activity assay utilizes 0.3 M PCA (perchloric acid) and malachite green reagent (8.27 mM sodium molybdate II, 0.33 mM malachite green oxalate, and 0.8 mM Triton X-100). To perform the assay, 10 μL of reaction is quenched in 90 μL of cold 0.3 M PCA. Phosphate standards are used so data can be converted to mM inorganic phosphate released.

When all reactions and standards have been quenched in PCA, 100 μL of malachite green reagent is added to the to relevant wells in e.g., a microtiter plate. The mixture is developed for 10-15 minutes and the plate is read at an absorbance of 650 nm. If phosphate standards were used, absorbance readings can be converted to mM Pi and plotted over time.

### Identifying Specific Modulators of Microtubule Binding.

As explained above, it was a discovery of this invention that small organic molecules can specifically modulate kinesin activity without mimicking the binding of either microtubules or ATP to the kinesin motor protein. The molecules thus are not competitive inhibitors of microtubule and ATP binding. This is a previously unknown mechanism of kinesin (or other motor, e.g., myosin or dynein) inhibition. Thus, in one embodiment, this invention provides methods of identifying such kinesin motor protein inhibitors that do not specifically block the microtubule or ATP binding sites. It is also expected that some small organic molecules will facilitate interactions at the microtubule binding site and similar assays can be used to identify such enhancers of kinesin motor activity.

Such specific inhibitors are characterized by the fact that they are not competitively inhibited by, and do not competitively inhibit, binders of the microtubule binding site and binders at the ATPase site. In one embodiment, this invention therefor provides methods of identifying compounds, especially small organic molecules which change kinesin motor activity only when the motor protein is bound to microtubules. The methods involve screening the “test” compound's ability to competitively inhibit binding of a moiety (e.g., ATP or an ATP analogue) at the ATPase site, screening the same compound's ability to competitively inhibit binding of a moiety (e.g., a microtubule) at the microtubule binding at the microtubule binding site, and finally, comparing the compound's inhibitory activity in the ATPase assay in the presence and absence of microtubules.

Methods of identifying competitive inhibition are well known to those of skill in the art. Briefly, in the classical Michaelis-Menton model of enzyme kinetics, competitive inhibition is easily recognized experimentally because the percent inhibition at a fixed inhibitor concentration is decreased by increasing the substrate concentration. Thus, where the compound competitively inhibits binding at the microtubule binding site, increasing the microtubule concentration at a fixed concentration of test compound can restore the original (inhibitor-free) maximal rate of reaction (V max). Conversely, where competition is non-competitive increasing the substrate concentration will not restore the maximal rate of reaction (Vmax). Assays for specific inhibition at the microtubule binding site are illustrated in example 1. For a detailed discussion of analysis of reaction kinetics to recognize competitive, noncompetitive and uncompetitive inhibition, see, e.g., Lehninger (1975) Biochemistry Worth Pub., Inc. New York, N.Y. This is the same paragraph as on p 4!

### High Throughput Screening

Conventionally, new chemical entities with useful properties are generated by identifying a chemical compound (called a “lead compound”) with some desirable property or activity, creating variants of the lead compound, and evaluating the property and activity of those variant compounds. However, the current trend is to shorten the time scale for all aspects of drug discovery. Because of the ability to test large numbers quickly and efficiently, high throughput screening (HTS) methods are replacing conventional lead compound identification methods.

In one preferred embodiment, high throughput screening methods involve providing a library containing a large number of potential therapeutic compounds (candidate compounds). Such “combinatorial chemical libraries” are then screened in one or more assays, as described herein, to identify those library members particular chemical species or subclasses) that display a desired characteristic activity. The compounds thus identified can serve as conventional “lead compounds” or can themselves be used as potential or actual therapeutics.

Devices for the preparation of combinatorial libraries are commercially available (see, e.g., 357 MPS, 390 MPS, Advanced Chem Tech, Louisville, Kent., Symphony, Rainin, Woburn, Mass., 433A Applied Biosystems, Foster City, Calif., 9050 Plus, Millipore, Bedford, Mass.).

A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.) which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

Any of the assays for anti-kinesin motor activity described herein are amenable to high throughput screening. As described above, the adocia-derived compounds may be screened for anti-kinesin motor activity in binding assays, motility assays, or assays for anti-mitotic activity.

High throughput systems for such screening are well known to those of skill in the art. Thus, for example, U.S. Pat. No. 5,559,410 discloses high throughput screening methods for protein binding, while U.S. Pat. Nos. 5,576,220 and 5,541,061 disclose high throughput methods of screening for ligand/antibody binding.

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols the various high throughput. Thus, for example, Zymark Corp. provides technical bulletins describing screening systems for detecting the modulation of gene transcription, ligand binding, and the like.

### EXAMPLES

Examples provided are intended to assist in a further understanding of the invention. Particular materials employed, species and conditions are intended to be illustrative of the invention and not limiting of the reasonable scope thereof.

### Assays

The compounds of the instant invention described in the Examples were tested by the assays described below and were found to have kinesin inhibitory activity. Other assays are known in the literature and could be readily performed by those of skill in the art (see, for example, PCT Publication WO 01/30768, May 3, 2001, pages 18-22).

### I. Kinesin ATPase In Vitro Assay

### Cloning and Expression of Human Poly-Histidine Tagged KSP Motor Domain (KSP(367H))

Plasmids for the expression of the human KSP motor domain construct were cloned by PCR using a pBluescript full length human KSP construct (Blangy et al., Cell, vol. 83, pp 1159-1169, 1995) as a template. The N-terminal primer 5′-GCAACGATTAATATGGCGTCGCAGCCAAATTCGTCTGCGAAG (SEQ.ID.NO.: 1) and the C-terminal primer 5′-GCAACGCTCGAGTCAGTGAT GATGGTGGTGATGCTGATTCACTTCAGGCTTATTCAATAT (SEQ.ID.NO.: 2) were used to amplify the motor domain and the neck linker region. The PCR products were digested with AseI and XhoI, ligated into the NdeI/XhoI digestion product of pRSETa (Invitrogen) and transformed into E. coli BL21 (DE3).

Cells were grown at 37° C. to an OD₆₀₀ of 0.5. After cooling the culture to room temperature expression of KSP was induced with 100 μM IPTG and incubation was continued overnight. Cells were pelleted by centrifugation and washed once with ice-cold PBS. Pellets were flash-frozen and stored −80° C.

### Protein Purification

Cell pellets were thawed on ice and resuspended in lysis buffer (50 mM K-HEPES, pH 8.0, 250 mM KCl, 0.1% Tween, 10 mM imidazole, 0.5 mM Mg-ATP, 1 mM PMSF, 2 mM benzimidine, 1× complete protease inhibitor cocktail (Roche)). Cell suspensions were incubated with 1 mg/ml lysozyme and 5 mM β-mercaptoethanol on ice for 10 minutes, followed by sonication (3×30 sec). All subsequent procedures were performed at 4° C. Lysates were centrifuged at 40,000×g for 40 minutes. Supernatants were diluted and loaded onto an SP Sepharose column (Phamacia, 5 ml cartridge) in buffer A (50 mM K-HEPES, pH 6.8, 1 mM MgCl₂, 1 mM EGTA, 10 μM Mg-ATP, 1 mM DTT) and eluted with a 0 to 750 mM KCl gradient in buffer A. Fractions containing KSP were pooled and incubated with Ni-NTA resin (Qiagen) for one hour. The resin was washed three times with buffer B (Lysis buffer minus PMSF and protease inhibitor cocktail), followed by three 15-minute incubations and washes with buffer B. Finally, the resin was incubated and washed for 15 minutes three times with buffer C (same as buffer B except for pH 6.0) and poured into a column. KSP was eluted with elution buffer (identical to buffer B except for 150 mM KCl and 250 mM imidazole). KSP-containing fractions were pooled, made 10% in sucrose, and stored at −80° C.

Microtubules are prepared from tubulin isolated from bovine brain. Purified tubulin (>97% MAP-free) at 1 mg/ml is polymerized at 37° C. in the presence of 10 μM paclitaxel, 1 mM DTT, 1 mM GTP in BRB80 buffer (80 mM K-PIPES, 1 mM EGTA, 1 mM MgCl₂ at pH 6.8). The resulting microtubules are separated from non-polymerized tubulin by ultracentrifugation and removal of the supernatant. The pellet, containing the microtubules, is gently resuspended in 10 μM paclitaxel, 1 mM DTT, 50 μg/ml ampicillin, and 5 μg/ml chloramphenicol in BRB80.

The kinesin motor domain (20 nM) is incubated with microtubules, ATP (1 mM 1:1 MgCl₂: Na-ATP), and compound at 23° C. in buffer containing 80 mM K-HEPES (pH 7.0), 1 mM EGTA, 1 mM DTT, 1 mM MgCl₂, and 50 mM KCl. The reaction is terminated by a 2-10 fold dilution with a final buffer composition of 80 mM HEPES and 50 mM EDTA (or, alternately, with a 1:1 addition of reaction volume to stop buffer(1.8M KCl and 50 mM EDTA)). Free phosphate from the ATP hydrolysis reaction is measured via a quinaldine red/ammonium molybdate assay by adding a 1.5 times volume of quench C (e.g., to a mixture of 401 reaction volume+40 μl stop buffer is then added 120 μl quench C). Quench A contains 0.1 mg/ml quinaldine red and 0.14% polyvinyl alcohol; quench B contains 12.3 mM ammonium molybdate tetrahydrate in 1.15 M sulfuric acid. Quench C is a 2:1 ratio of quench A:quench B The reaction is incubated for 5-10 minutes at 23° C., and the absorbance of the phospho-molybdate complex is measured at 540 nm.

The in vitro substrate competitive profiles of the compounds described in Table 1 below were determined using a Michaelis-Menton analysis with the assay described above, but varying assay concentrations of one of the substrates (ATP or microtubule) in the presence of excess of the second substrate. Thus, the concentration of ATP was varied between 4 μM and 500 μM, in the presence of 2 μM of microtubules. And the concentration of microtubules was varied between 0.01 μM and 5 μM, in the presence of 1 mM of ATP.

In order to identify compounds useful in the methods of the instant invention, the assay described above was then performed in the presence (0.5 μM) and absence of microtubules. Results are shown in Tables 1 and 2. Compounds that are commercially available are noted by the source and catalog number.

TABLE 1

| | KSP ATPase | Competitive | Competitive |
|---|---|---|---|
| Structure | IC50 (nM) | with ATP? | with MT? |
| 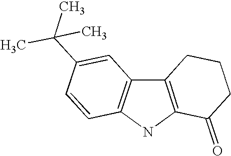 1SPECS AG-690/37024143 | 597 | no | no |
| 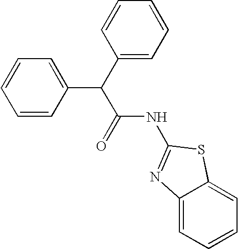 2Asinex BAS0148158 | 1087 | no | no |
| 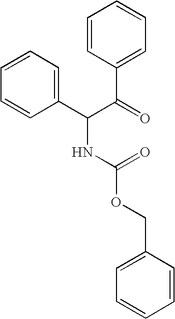 3 | 2365 | no | no |
| 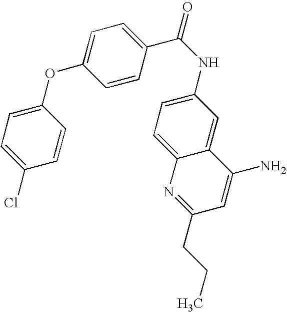 4 | 3233 | no | no |
| 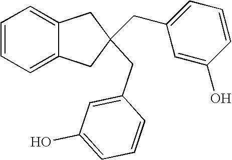 5 | 5873 | no | no |
| 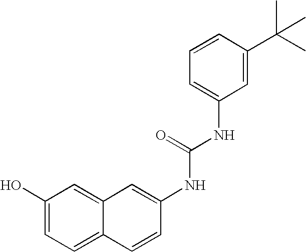 6 | 1896 | no | no |
| 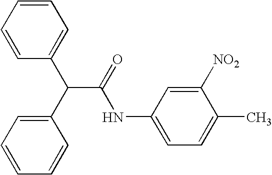 7Asinex BAS0394630 | 3849 | no | no |
| 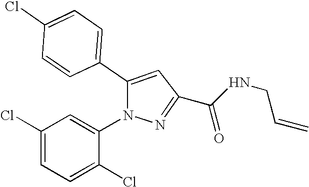 Bionet 4N-6125 | 5037 | no | no |
| 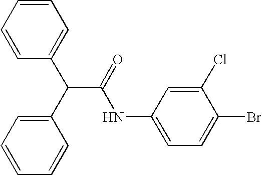 9Chembridge 6177492 | 692 | no | no |
| 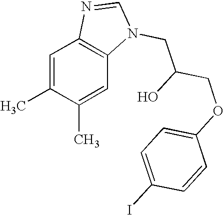 10ChemDiv 3772-4389 | 4745 | no | no |
| 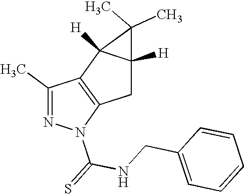 11Interbioscreen STOCK 1N-27769 | 1232 | no | no |
| 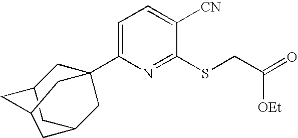 12Interbioscreen STOCK 1S-62448 | 556 | no | |
| 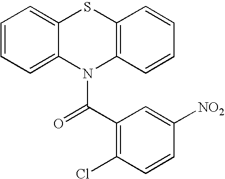 13Interbioscreen STOCK 2S-21956 | 864 | no | no |
| 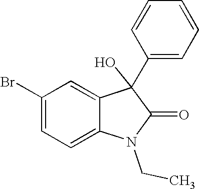 14Merlin Synthesis MS2630 | 3922 | no | no |
| 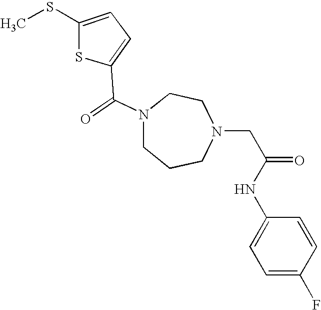 15Tripos 1518-04640 | 7021, 6750 | no | no |

TABLE 2

| | KSP ATPase −/+ Microtubules | |
|---|---|---|
| | using standard KSP(ADP) | |
| | KSP ATPase | KSP ATPase |
| | IC50 (nM) | IC50 (nM) |
| Structure | [MT] = 0.5 uM | [MT] = 0 uM |
| 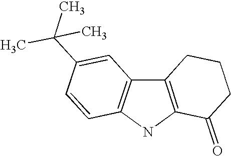 1 | 619, 685 | >50000,>50000 |
| 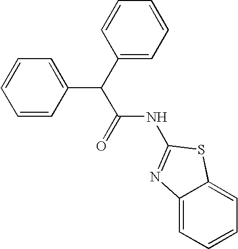 2 | 1396, 2154 | >50000,>50000 |
| 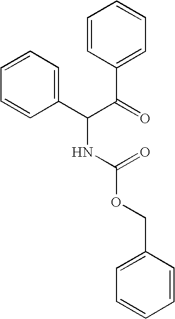 3 | 4052, 2943 | >50000,>50000 |
| 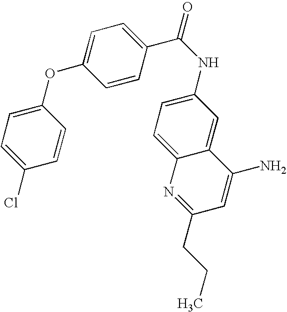 4 | 2554, 3428 | >50000,>50000 |
| 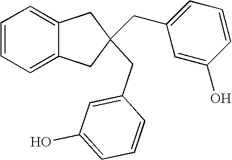 5 | 7744 | >50000 |
| 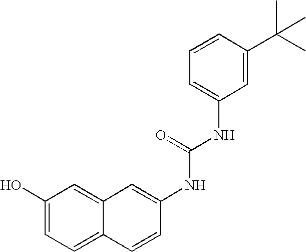 6 | 2779 | >50000 |
| 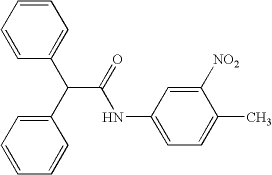 7Asinex BAS0394630 | 3690, 6293 | >50000,>50000 |
| 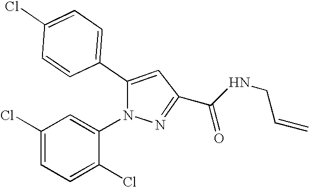 8Bionet 4N-6125 | 3096, 5543 | >50000,>50000 |
| 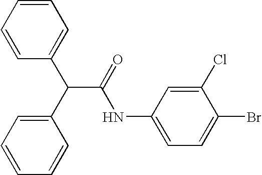 9Chembridge 6177492 | 647 | >50000 |
| 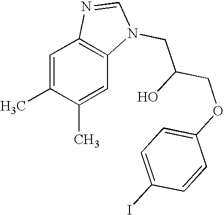 10ChemDiv 3772-4389 | 5042 | >50000 |
| 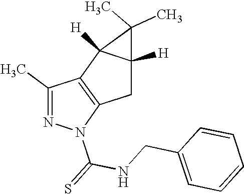 11Interbioscreen STOCK 1N-27769 | 1823, 1356 | >50000,>50000 |
| 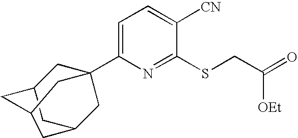 12Interbioscreen STOCK 1S-62448 | 1153 | >50000 |
| 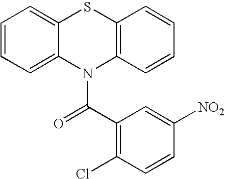 13Interbioscreen STOCK 2S-21956 | 2529, 4516 | >50000,>50000 |
| 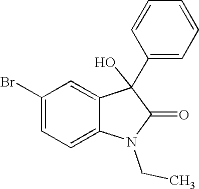 14Merlin Synthesis MS2630 | 8925, 4601 | >50000,>50000 |
| 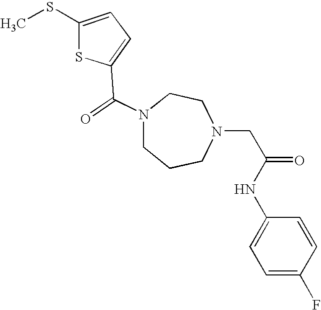 15Tripos 1518-04640 | 8883 | >50000,>50000 |

### II. Cell Proliferation Assay

Cells are plated in 96-well tissue culture dishes at densities that allow for logarithmic growth over the course of 24, 48, and 72 hours and allowed to adhere overnight. The following day, compounds are added in a 10-point, one-half log titration to all plates. Each titration series is performed in triplicate, and a constant DMSO concentration of 0.1% is maintained throughout the assay. Controls of 0.1% DMSO alone are also included. Each compound dilution series is made in media without serum. The final concentration of serum in the assay is 5% in a 200 μL volume of media. Twenty microliters of Alamar blue staining reagent is added to each sample and control well on the titration plate at 24, 48, or 72 hours following the addition of drug and returned to incubation at 37° C. Alamar blue fluorescence is analyzed 6-12 hours later on a CytoFluor II plate reader using 530-560 nanometer wavelength excitation, 590 nanometer emission.

A cytotoxic EC₅₀ is derived by plotting compound concentration on the x-axis and average percent inhibition of cell growth for each titration point on the y-axis. Growth of cells in control wells that have been treated with vehicle alone is defined as 100% growth for the assay, and the growth of cells treated with compounds is compared to this value. Proprietary in-house software is used to calculate percent cytotoxicity values and inflection points using logistic 4-parameter curve fitting. Percent cytotoxicity is defined as:

% cytotoxicity:(Fluorescence_{control})−(Flourescenceₛₐₘₚₗₑ)×100×(Fluorescence_{control})⁻¹

The inflection point is reported as the cytotoxic EC₅₀.

### III. Evaluation of Mitotic Arrest and Apoptosis by FACS

FACS analysis is used to evaluate the ability of a compound to arrest cells in mitosis and to induce apoptosis by measuring DNA content in a treated population of cells. Cells are seeded at a density of 1.4×10⁶ cells per 6 cm² tissue culture dish and allowed to adhere overnight. Cells are then treated with vehicle (0.1% DMSO) or a titration series of compound for 8-16 hours. Following treatment, cells are harvested by trypsinization at the indicated times and pelleted by centrifugation. Cell pellets are rinsed in PBS and fixed in 70% ethanol and stored at 4° C. overnight or longer.

For FACS analysis, at least 500,000 fixed cells are pelleted and the 70% ethanol is removed by aspiration. Cells are then incubated for 30 min at 4° C. with RNase A (50 Kunitz units/ml) and propidium iodide (50 μg/ml), and analyzed using a Becton Dickinson FACSCaliber. Data (from 10,000 cells) is analyzed using the Modfit cell cycle analysis modeling software (Verity Inc.).

An EC₅₀ for mitotic arrest is derived by plotting compound concentration on the x-axis and percentage of cells in the G2/M phase of the cell cycle for each titration point (as measured by propidium iodide fluorescence) on the y-axis. Data analysis is performed using the SigmaPlot program to calculate an inflection point using logistic 4-parameter curve fitting. The inflection point is reported as the EC₅₀ for mitotic arrest. A similar method is used to determine the compound EC₅₀ for apoptosis. Here, the percentage of apoptotic cells at each titration point (as determined by propidium iodide fluorescence) is plotted on the y-axis, and a similar analysis is carried out as described above.

### IV. Immunofluorescence Microscopy to Detect Monopolar Spindles

Methods for immunofluorescence staining of DNA, tubulin, and pericentrin are essentially as described in Kapoor et al. (2000) J. Cell Biol. 150: 975-988. For cell culture studies, cells are plated on tissue culture treated glass chamber slides and allowed to adhere overnight. Cells are then incubated with the compound of interest for 4 to 16 hours. After incubation is complete, media and drug are aspirated and the chamber and gasket are removed from the glass slide. Cells are then permeabilized, fixed, washed, and blocked for nonspecific antibody binding according to the referenced protocol. Paraffin-embedded tumor sections are deparaffinized with xylene and rehydrated through an ethanol series prior to blocking. Slides are incubated in primary antibodies (mouse monoclonal anti-α-tubulin antibody, clone DM1A from Sigma diluted 1:500; rabbit polyclonal anti-pericentrin antibody from Covance, diluted 1:2000) overnight at 4° C. After washing, slides are incubated with conjugated secondary antibodies (FITC-conjugated donkey anti-mouse IgG for tubulin; Texas red-conjugated donkey anti-rabbit IgG for pericentrin) diluted to 15 μg/ml for one hour at room temperature. Slides are then washed and counterstained with Hoechst 33342 to visualize DNA. Immunostained samples are imaged with a 100× oil immersion objective on a Nikon epifluorescence microscope using Metamorph deconvolution and imaging software.

### Example 1

### tert-Butyl phenyl(tolylsulfonyl)methylcarbamate

A round-bottomed flask fitted with an addition funnel and an overhead mechanical stirrer was charged with p-toluenesulfinic acid, sodium salt (1.5 eq) followed by tert butylcarbamate (1.5 eq). The flask was then charged with acetonitrile (500 mL) and the contents stirred and placed under a positive pressure of nitrogen. To the resulting slurry was added benzaldehyde (1.0 eq) in one portion. The resulting mixture was then cooled to 10° C. using an ice bath. To the addition funnel was added chlorotrimethylsilane (TMSCl) (2.0 eq) and this was slowly added to the reaction mixture to maintain an internal temperature below 25° C. (total addition time was 15 min). After complete addition of the TMSCl, the reaction was allowed to warm to room temperature (23° C.). The reaction was then monitored by HPLC until completion (24 hours). To the heterogeneous mixture was added water (500 mL) and the resulting suspension was stirred for 30 min. The solids were isolated by filtration and the filter cake was washed with water (100 mL). The solid was dried in a vacuum oven at 50° C. at 30 torr for 24 hours to give the product as a fluffy white solid.

¹H NMR (400 MHz, CDCl₃) δ; 7.82-7.78 (d, 2H, J=8.4 Hz), 7.50-7.40 (m, 5H), 7.32-7.38 (d, 2H, J=8.3 Hz), 6.01-5.90 (d, 1H, J=10.4 Hz), 5.86-5.79 (d, 1H, J=10.3 Hz), 2.45-2.40 (s, 3H), 1.40-1.20 (br, 9H); ¹³C NMR (100 MHz, CDCl₃) δ 154.0, 144.9, 133.8, 130.0, 129.7, 129.6, 129.4, 128.8, 128.6, 81.0, 73.8, 27.9, 21.5.

### tert-Butyl N-(α-tosylbenzyl)carbamate

¹H NMR (400 MHz, CDCl₃) δ 7.82-7.78 (d, 2H, J=8.4 Hz), 7.50-7.40 (m, 5H), 7.32-7.38 (d, 2H, J=8.3 Hz), 6.01-5.90 (d, 1H, J=10.4 Hz), 5.86-5.79 (d, 1H, J=10.3 Hz), 2.45-2.40 (s, 3H), 1.40-1.20 (br, 9H); ¹³C NMR (100 MHz, CDCl₃) δ 154.0, 144.9, 133.8, 130.0, 129.7, 129.6, 129.4, 128.8, 128.6, 81.0, 73.8, 27.9, 21.5.

### N-(2-Oxo-1,2-diphenyl-ethyl)-carbamic acid tert-butyl ester

The product was isolated from the crude reaction mixture by crystallization from ethyl acetate as small white needles;

mp 113-114° C.; ¹H NMR (400 MHz, CDCl₃) δ 8.01-7.90 (d, 2H, J=7.6 Hz), 7.53-7.46 (t, 1H, J=7.6 Hz), 7.43-7.34 (t, 4H, J=7.6 Hz), 7.32-7.20 (m, 3H), 6.35-6.25 (d, 1H, J=7.2 Hz), 6.12-6.00 (d, 1H, J=7.2 Hz), 1.50-1.38 (br, 9H); ¹³C NMR (100 MHz, CDCl₃) δ 196.1, 155.2, 137.4, 134.5, 133.5, 129.0, 128.5, 128.2, 128.0, 79.8, 59.7, 28.3; Anal. Calcd for C₁₉H₂₁NO₃: C, 73.29; H, 6.80; N, 4.50. Found: C, 72.91; H, 6.76; N, 4.42.

### N-(2-Oxo-1,2-diphenyl-ethyl)-carbamic acid benzyl ester (Compound 3)

Employing the above described procedure, but substituting benzyl carbamate for tert-butyl carbamate, the title compound was prepared.

### Example 2

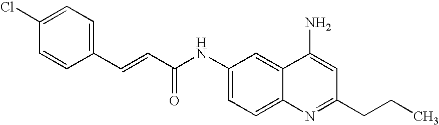

### (2E)-N-(4-Amino-2-propylquinolin-6-yl)-3-(4-chlorophenyl)prop-2-enamide

### Step 1: Preparation of ethyl (2E)- and (2Z)-3-{[4-(acetylamino)-phenyl]amino}hex-2-enoate

A mixture of N-(4-aminophenyl)acetamide (9.7 g, 65 mmol), ethyl 3-oxohexanoate (10 g, 65 mmol) and 2 drops conc. HCl in 30 mL ethanol was heated at reflux overnight. After approximately 18 h, the reaction mixture was cooled to r.t. and the solids collected by filtration. The solids were washed with methanol and air dried to afford the crude product as a solid, which was used without further purification in the subsequent reaction.

### Step 2: Preparation of N-(4-hydroxy-2-propylquinolin-6-yl)acetamide

The crude product (9.0 g) described in Step 1 was mixed with 50 mL of diphenylether. The mixture was heated with a heating mantle at 260° for 2 h then cooled to r.t. The resulting solid was collected by filtration, washed with EtOAc to give a grey solid, which was used directly in the next step.

### Step 3: Preparation of N-(4-methoxy-2-propylquinolin-6-yl)acetamide

The crude product (5.9 g) described in Step 2 and dimethylsulfate (4.6 mL, 48 mmol) were mixed in toluene and heated at reflux for 2.5 h. The reaction mixture was cooled to r.t. and the precipitate was collected by filtration. The solids were washed with toluene, air dried then added to a mixture of 50 mL 1N aq. NaOH and 100 mL EtOAc. The solids were filtered and washed with EtOAc. The filtrate was transferred to a separatory funnel and the layers separated. The aqueous layer was extracted with excess EtOAc. The organic layers were combined and the solvent removed under vacuum to afford the product as a yellow solid, MS: m/z 259 (MH⁺).

### Step 4: Preparation of N-(4-amino-2-propylquinolin-6-yl)acetamide

An intimate mixture of the crude product (4.0 g) described in Step 3 and ammonium acetate (40 g, 52 mmol) were heated at 140° to 150° for 4 h. The reaction mixture was cooled to r.t. to provide the crude product which used immediately without further purification.

### Step 5: Preparation of 2-propylquinoline-4,6-diamine

To the above crude reaction mixture described in Step 4 was added 30 mL water and 40 mL conc. HCl. The resulting mixture was heated at 90° for 5 h then cooled to r.t. The remaining precipitate was collected by filtration. The aqueous filtrate was concentrated under vacuum then made basic by addition of aq. sodium hydroxide. The aqueous mixture was transferred to a separatory funnel and extracted with excess EtOAc. The organic layers were combined, dried with a drying agent and the solvent removed under vacuum to afford a solid, MS: m/z 202 (MH⁺).

### Step 6: Preparation of (2E)-3-(4-chlorophenyl)prop-2-enoyl chloride

To a solution of (2E)-3-(4-chlorophenyl)prop-2-enoic acid (2.0 g, 11 mmol) in 50 mL methylene chloride was added oxalyl chloride (1.05 mL, 12.1 mmol) and N,N-dimethylformamide (0.05mL, 0.6 mmol). The resulting mixture was stirred at r.t. for 6 h. The solvent was removed under vacuum. The resulting solid was diluted with hexanes and the solvent removed under vacuum to provide an off-white solid, which was used without further purification.

### Step 7 Preparation of (2E)-N-(4-Amino-2-propylquinolin-6-yl)-3-(4-chlorophenyl)prop-2-enamide

To a solution of the product described in Step 5 (60 mg, 0.3 mmol) in 1.5 mL HOAc was added the product described in Step 6 (64 mg, 0.32 mmol). The resulting mixture was stirred at r.t. for 6 h then the solvent removed under vacuum. The residue was purified by preparative TLC eluting with chloroform/2N ammonia in methanol (9/1) to afford the product, MS: m/z 366 (MH⁺).

Following a procedure similar to that described above for Example 3, the following compound was prepared from 2-propylquinoline-4,6-diamine:

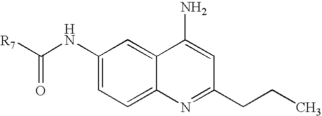

| | | Parent Ion |
|---|---|---|
| Compound No. | R₇ | (MH+) m/z |
| 4 | 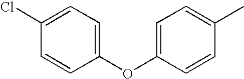 | 432 |

### Example 3

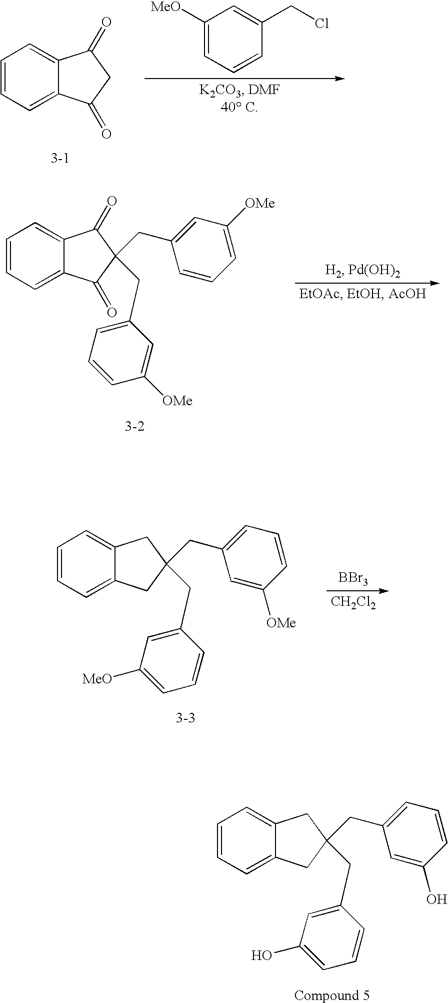

Step 1: A mixture of 1,3-indandione (1.71 mmol), potassium carbonate (6.29 mmol), and DMF (5 mL) was heated to 40° C. A deep red color was obtained. After which, 3-methoxybenzyl chloride (5.16 mmol) was added. The resulting mixture was stirred at 40° C. for 14 d. The mixture was allowed to cool and was aged at rt for an additional 6 d. The reaction was partitioned between water and ethyl acetate. The organic layer was washed with brine, dried (MgSO₄), filtered, and concentrated. The residue was purified via preparative plate chromatography (4×1500% plates) eluting with hexane:ethyl acetate (6:1). A second chromatography (2×1500μ plates) eluting with hexane:ethyl acetate (2:1) yield the desired compound, 3-2.

Step 2: Bis alkylated indandione 3-2 (0.44 mmol) was dissolved in 1:1 ethyl acetate:ethanol (10 mL). Glacial acetic acid (0.1 mL) was added followed by Pd(OH)₂ (78 mg, ˜50% H₂O). The vessel was evacuated and purged with nitrogen (3×) and then hydrogen (3×). The reaction stirred at rt under a hydrogen balloon for 2 d and was filtered though a pad of celite rinsing with ethyl acetate. The filtrate was concentrated and purified via preparative plate chromatography (4×1000μplates) eluting with hexane:ethyl acetate (6:1) to give 3-3.

Step 3: Indane 3-3 (0.05 mmol) was dissolved in anhydrous methylene chloride (2 mL) and cooled to 0° C. under nitrogen. A solution of boron tribromide (0.34 mmol) in methylene chloride (1.0 M) was added. After 2 h, the reaction was quenched with saturated aqueous sodium bicarbonate. The mixture was partitioned between water and ethyl acetate. The organic layer was washed with brine, dried (Na₂SO₄), filtered, and concentrated. The residue was concentrated and purified via preparative plate chromatography (1×1000μ plate) eluting with hexane:ethyl acetate (2:1) to give Compound 5.

### Example 4

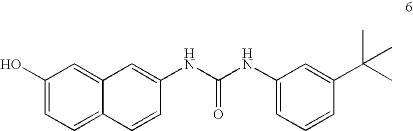

### Step 1: 7-Methoxy-2-naphthoic acid

To a solution of methyl 7-methoxy-2-naphthoate [Synlett, (1991), (6), p-405] (9.5 g, 44 mmol) in ethanol (100 ml) was added a solution of potassium hydroxide (7.4 g, 132 mmol) in water (100 ml), and the resulting mixture heated at reflux for 16 hours. The mixture was cooled and the ethanol remove in vacuo. The residue was diluted with more water (100 ml) and extracted with dichloromethane (3×100 ml). The aqueous layer was acidified by the addition of 5N HCl and extracted with dichloromethane (3×100 ml). The combined extracts were dried over Na₂SO₄, filtered and evaporated to give the title compound as a white solid. ¹H NMR (400 MHz, DMSO) 3.91 (3H, s), 7.31 (1H, dd, J 9.0 and 2.6), 7.54 (1H, d, J2.6), 7.84 (1H, dd, J 8.4 and 1.6), 7.92 (2H, t, J 8.4 and 8.1), 8.53 (1H, d, J 0.6), 13.00 (1H, s).

### Step 2: 7-Hydroxy-2-naphthoic acid

A suspension of 7-methoxy-2-naphthoic acid (7.0 g, 34.6 mmol) and 48% hydrobromic acid (100 ml) was heated at reflux for 14 hours. The cooled mixture was evaporated to dryness, and the resulting solid triturated with acetone, filtered and dried to give the title compound as a pale brown solid.

¹H NMR (400 MHz, DMSO) 7.23 (1H, dd, J 8.9 and 2.5), 7.31 (1H, d, J 2.3), 7.76 (1H, dd, J 8.5 and 1.6), 7.86 (2H, t, J 8.0), 8.39 (1H, s), 9.95 (1H, s), 12.95 (1H, brs).

### Step 3: Benzyl 7-(benzyloxy)-2-naphthoate

To a solution of 7-hydroxy-2-naphthoic acid (7.15 g, 38 mmol) in anhydrous N,N-dimethylformamide (100 ml) was added potassium carbonate (15.75 g, 114 mmol), followed by benzyl bromide (11.3 ml, 95 mmol). The resulting mixture was stirred at room temperature for 14 hours. The mixture was poured into water (500 ml) and extracted with ethyl acetate (3×100 ml), the combined ethyl acetate layers were washed with water (2×200 ml), sat. NaCl (100 ml), dried over Na₂SO₄, filtered and evaporated to give the title compound. ¹H NMR (400 MHz, DMSO) 5.24 (2H, s), 5.41 (2H, s), 7.33-7.41 (7H, m), 7.43-7.46 (4H, m), 7.70 (1H, d, J 2.5), 7.86 (1H, dd, J 8.5 and 1.7), 7.96 (2H, t, J 9.8 and 9.1), 8.55 (1H, d, J 0.5).

### Step 4: 7-(Benzyloxy)-2-naphthoic acid

To a suspension of benzyl 7-(benzyloxy)-2-naphthoate (8.77 g, 23.8 mmol) in ethanol (100 ml) was added a solution of potassium hydroxide (5.34 g, 95.2 mmol) in water (100 ml), and the resulting mixture heated at reflux for 2 hours. The mixture was cooled and the ethanol removed in vacuo. The residue was diluted with more water (100 ml) and extracted with dichloromethane (2×100 ml). The aqueous layer was acidified by the addition of conc. HCl, and the mixture extracted with 10% methanol in dichloromethane (3×150 ml). The combined organic layers were dried over Na₂SO₄, filtered and evaporated. The residue was triturated with acetone, filtered and dried to give the title compound as a light brown powder. ¹H NMR (360 MHz, DMSO) 5.26 (2H, s), 7.33-7.41 (4H, m), 7.53 (2H, d, J 7.1), 7.65 (1H, d, J 2.4), 7.85 (1H, dd, J 8.5 and 1.5), 7.92 (2H, m), 8.51 (1H, s), 13.03 (1H, s).

### Step 5: N-[7-(Benzyloxy)-2-naphthyl]-N′-(3-tert-butylphenyl)urea

To a suspension of 7-(benzyloxy)-2-naphthoic acid (500 mg, 1.8 mmol) in anhydrous toluene (15 ml) was added triethylamine (0.251 ml, 1.8 mmol), followed by diphenylphosphorylazide (0.388 ml, 1.8 mmol). The resulting mixture was heated at reflux for 1 hour, after which time 3-tert butylaniline (537 mg, 3.6 mmol) was added, and heating continued for a further 12 hours. The cooled reaction mixture was evaporated and the residue treated with methanol where upon a precipitate formed which was removed by filtration and dried to give the title compound as a pale orange solid. ¹H NMR (400 MHz, DMSO) 1.29 (9H, s), 5.22 (2H, s), 7.02 (1H, d, J 8.2), 7.05 (1H, dd, J 9.0 and 2.7), 7.21 (1H, t, J 7.8), 7.29-7.36 (4H, m), 7.41 (2H, t, J 7.4 and 7.0), 7.50-7.53 (3H, m), 7.71 (1H, d, J 9.0), 7.73 (1H, d, J 8.6), 8.04 (1H, d, J 2.0), 8.69 (1H, s), 8.78 (1H, s).

### Step 6: N-(3-tert-butylphenyl)-N-(7-hydroxy-2-naphthyl)urea

To a nitrogen flushed solution of N-[7-(benzyloxy)-2-naphthyl]-N′-(3-tert-butylphenyl)urea (424 mg, 1 mmol) in a mixture of ethyl acetate (80 ml) and methanol (20 ml) contained in a Parr flask was added 10% Palladium on carbon (100 mg), and the resulting mixture hydrogenated at 50 psi for 2 hours. The catalyst was removed by filtration, and the filtrate evaporated to dryness. The oily residue was crystallized from dichloromethane, filtered and dried to give the title compound as a white solid. ¹H NMR (400 MHz, DMSO) 1.29 (9H, s), 6.91 (1H, dd, J 9.0 and 2.3), 6.99-7.02 (2H, m), 7.19-7.24 (2H, m), 7.30 (1H, dd, J 8.2 and 1.2), 7.49 (1H, m), 7.64 (1H, d, J 8.6), 7.67 (1H, d, J 8.6), 7.87 (1H, d, J 1.6), 8.69 (1H, s), 8.73 (1H, s), 9.62 (1H, s); m/z (ES⁺) 335 (M+H⁺).

What is claimed is:

1. A method of identifying a compound that specifically modulates the activity of a kinesin motor protein bound to a microtubule, said kinesin motor protein having a microtubule binding site and a kinesin ATPase binding site, said method comprising the steps of:
a) assaying for competitive inhibition of said motor protein by said compound at said kinesin ATPase binding site;
b) assaying for competitive inhibition of said motor protein by said compound at said microtubule binding site;
c) assaying for inhibition of said motor protein by said compound in the absence of microtubules;
d) assaying for inhibition of said motor protein by said compound in the presence of microtubules;
e) identifying a compound as a kinesin-bound-to-microtubule modulator when said compound inhibits said motor protein activity in the presence of microtubules, is not a competitive modulator at said microtubule binding site and at said kinesin ATPase binding site, and does not inhibit said motor protein activity when microtubules are absent.

2. The method according to claim 1 wherein the compound is a polypeptide.

3. The method according to claim 1 wherein the compound is a small organic molecule.

4. The method according to claim 3 wherein the small organic molecule is a part of a combinatorial library when contacted with the kinesin motor protein.

5. The method according to claim 1 wherein the kinesin motor protein is KSP.

6. A method of modulating kinesin motor activity, said method comprising contacting a kinesin motor, said kinesin motor protein having a microtubule binding site and a kinesin ATPase binding site, with a small organic molecule that inhibits said motor protein activity in the presence of microtubules, is not a competitive modulator at said microtubule binding site and at said kinesin ATPase binding site, and does not inhibit said motor protein activity when microtubules are absent.

7. The method according to claim 6 wherein the kinesin motor protein is KSP.

8. A method of modulating cellular growth in an organism, said method comprising administering to said organism a composition comprising a pharmaceutically acceptable carrier and a compound in a quantity sufficient to alter said cellular growth in an organism, said compound identified by the method according to claim 1.

9. The method of claim 8, wherein said organism is an animal.

10. The use of the compound identified by the method according to claim 1 for the preparation of a medicament useful for the treatment or prevention of cancer.

11. The use of the compound selected from: 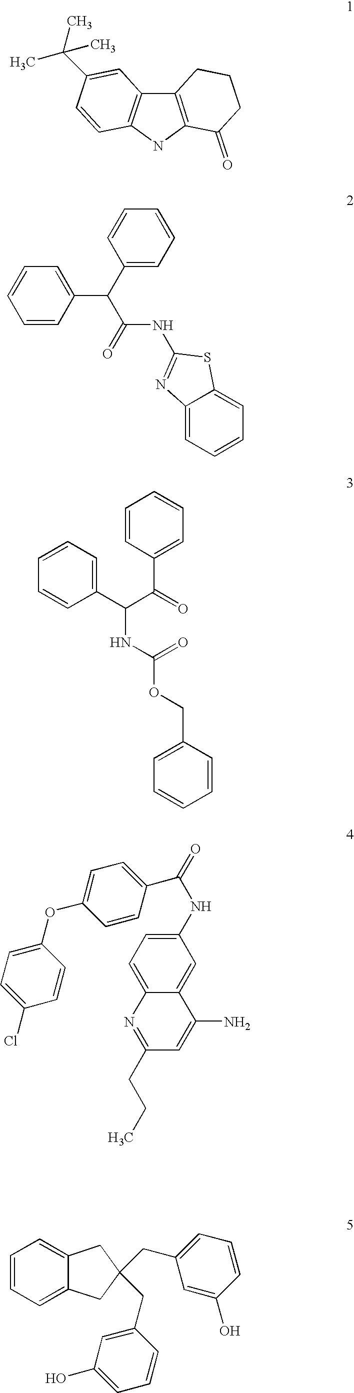 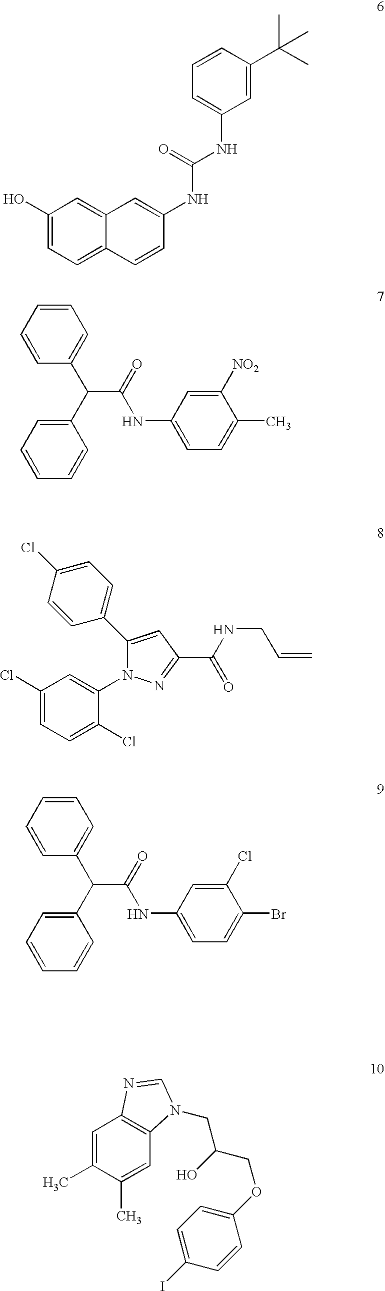 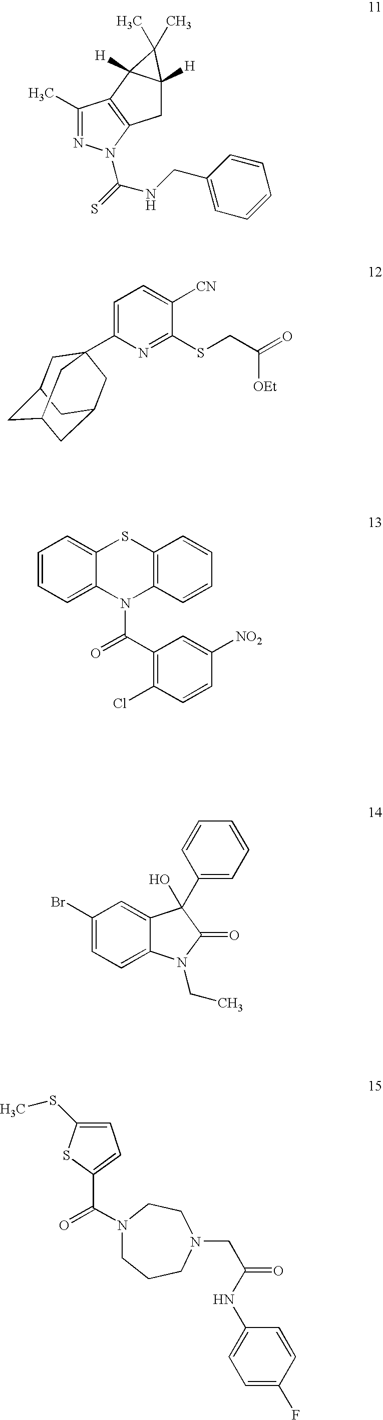
or a pharmaceutically acceptable salt thereof, for the preparation of a medicament useful for the treatment or prevention of cancer.

12. A compound selected from: 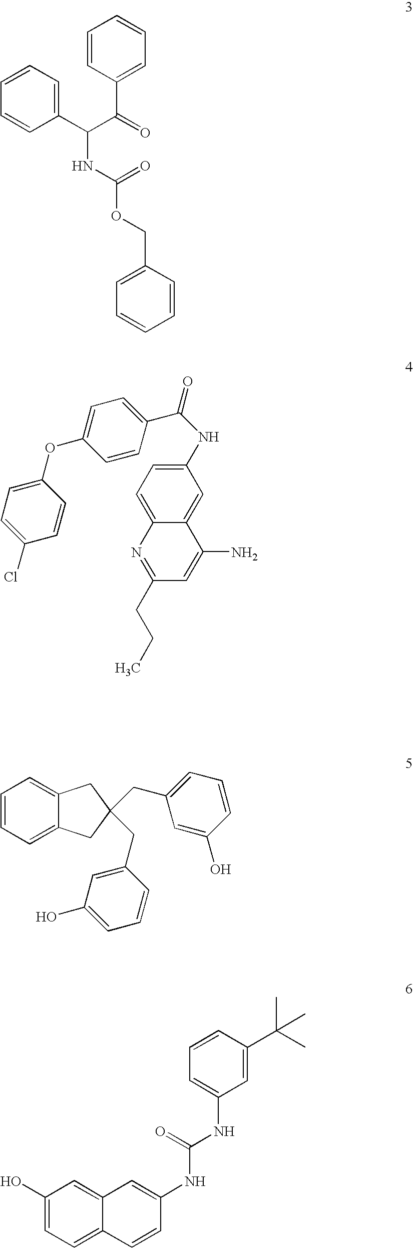
or a pharmaceutically acceptable salt thereof.
